(19) United States
(12) Patent Application Publication
(10) Pub. No.: US 2020/0397816 A1
(43) Pub. Date: Dec. 24, 2020

(54) METHODS TO TREAT INFLAMMATORY AND SMOOTH MUSCLE CELL PROLIFERATION DISORDERS

(71) Applicant: THE TRUSTEES OF INDIANA UNIVERSITY, Bloomington, IN (US)

(72) Inventors: Margaret Arlene SCHWARZ, Bristol, IN (US); Daniel Dong LEE, South Bend, IN (US); Arambage Dushani RANASINGHE, Notre Dame, IN (US)

(21) Appl. No.: 16/911,369

(22) Filed: Jun. 24, 2020

Related U.S. Application Data
(60) Provisional application No. 62/929,623, filed on Nov. 1, 2019.
(60) Provisional application No. 62/865,578, filed on Jun. 24, 2019.

(51) Int. Cl. A61K 33/16; A61P 9/10

(52) U.S. Cl. CPC A61K 33/16; A61P 9/10

(57) ABSTRACT

Disclosed are methods to treat inflammatory disorders and smooth muscle cell proliferation disorders in a subject by administering to the subject in need thereof an endothelial monocyte activating polypeptide II (EMAP II) inhibitor.

### CROSS REFERENCE TO RELATED APPLICATIONS

This application claims priority to the following: U.S. Provisional Application Ser. No. 62/865,578 filed Jun. 24, 2019, and U.S. Provisional Patent Application No. 62/929,623 filed on Nov. 1, 2019, the disclosures of which are hereby expressly incorporated by reference in their entirety.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 5.8 kilobytes acii (text) file named “321770_ST25.txt,” created on Jun. 24, 2020.

### BACKGROUND OF THE DISCLOSURE

Ubiquitously found in mammalian cell membranes, sphingolipids constitute 10-20% of the membrane lipids. Recent studies emphasize sphingolipids as critical regulators of a plethora of biological processes and diseases.

Sphingosine, an 18-carbon amino alcohol with an unsaturated hydrocarbon chain serves as the backbone of many sphingolipids. Phosphorylation of sphingosine by Sphingosine kinases (SphK1 and SphK2) generates the bioactive sphingolipid Sphingosine-1-Phosphate (S1P), a key mediator of various biological processes such as cell proliferation and growth, apoptosis, inflammation, angiogenesis and vascular integrity.

S1P is a classical signaling molecule involved in key steps of the stress response acting either as an intracellular secondary messenger or extracellular signaling molecule. Importantly, S1P extracellular signaling is cell-type and receptor dependent. Upon binding to one of five cell-specific G-protein coupled receptors, SIP receptor 1 (S1PR1)-S1P receptor 5 (S1PR5) can signal in part through the activation of the Janus tyrosine kinase/signal transducer and activators of transcription (JAK/STAT) signaling pathway. Upon stress or inflammation, a prototypical signal molecule (S1P) is induced and ‘Inside-out’ relocation of S1P initiates autocrine or paracrine signaling. The distinct and diverse functions of S1P require tight control of its abundance in different cell types and compartments. Regulation of S1P levels is through synthesis or breakdown by three enzymes: SphK that synthesizes S1P through phosphorylation of sphingosine, while S1P lyase (SPL) irreversibly degrades SIP by cleaving the acyl chain and phosphohydrolases, reversing the action of SphK. Recent studies determined that altered S1P homeostasis contributes to progression of cardiovascular and pulmonary pathophysiological processes characterized by inflammation and arterial narrowing such as atherosclerosis, pulmonary hypertension, and lung disease of prematurity. For example, in chronic inflammatory disease processes such as atherosclerosis, S1P serum levels were identified as a robust biomarker of severe disease progression. Additionally, S1P levels were elevated in lung tissues of patients with pulmonary hypertension (PH) where increased transcription of SphK1 plays a critical role in disrupting the S1P homeostasis and increasing proliferation of perivascular smooth muscle cells (SMCs) promoting development of PH. Similarly, elevated levels of S1P in tracheal fluids of preterm infants is associated with lung disease of prematurity while conversely the knockdown or pharmacological inhibition of SphK1 attenuates BPD formation in mouse models (Harijith et al., 2013; Hendricks-Muñoz, Xu and Voynow, 2018). Importantly, during tissue injury and inflammation signaling molecules such as S1P, Fractalkine, and Endothelial Monocyte Activating Polypeptide II (EMAP II) recruit macrophages and induce secretion of proinflammatory cytokines such as IL-1β, IL6 and Tnfα. Therefore, the study of signaling and molecular interactions of these signaling molecules is pivotal for the pathological understanding of complex diseases.

For example, Bronchopulmonary Dysplasia (BPD) is a debilitating consequence of environmental stress acting on vulnerable underdeveloped lungs of prematurely born infants. BPD is characterized by arrested formation of alveolar-capillary networks known as alveolar simplification and pulmonary hypertension. Compelling evidence for a pro-inflammatory contribution of bioactive sphingolipids, including the sphingosine kinase (SphK) enzymatically-phosphorylated Sphingosine-1-phosphate (S1P), to the pathobiology of BPD warrants the identification of strategic regulatory mediators that effectively regulate S1P activation to minimize alveolar simplification and pulmonary hypertension.

Recent studies have characterized the action of S1P upon signaling and physiological functions, e.g., homing of lymphocyte and myeloid cells, STAT3 signaling. However, there is a lack of knowledge regarding the regulating factors upstream of S1P, and no previous teaching that connects EMAP II to S1P signaling. Applicant is the first to describe the signaling connection between EMAP II and regulation of S1P release. Applicant is also the first to recognize the impact of EMAP II, operating through an S1P mediated mechanism, on pulmonary artery smooth muscle proliferation (PASMC).

EMAP II is the 23 kDa cleaved C-terminal peptide of the pervasively expressed 34-kDa intracellular protein Aminoacyl tRNA synthetase complex Interacting Multifunctional Protein 1 (AIMPI, also known as p43/proEMAP II: SEQ ID NO: 1) that functions as one of three scaffold proteins for the multi-synthetase complex (MSC) that mediate protein translation. It is the mature 23 kDa form (ENAP II; SEQ ID NO: 2) that is released from the cells and once EMAP II is released, it functions as a pro-inflammatory promiscuous signaling cytokine that predominately signals through tyrosine kinases. Accordingly, S1P mediation of inflammation and smooth muscle cell (SMC) proliferation may be strategically linked to an EMAP II initiated signaling cascade.

As disclosed herein the role of EMAP II in modulating S1P homeostasis and key intermediates in the SphK1/S1P signaling pathway was investigated. EMAP II was identified as a strategic early regulator of SphK1/S1P where it signals through ERK1/2 and EGR1 in macrophages and SMCs. Furthermore, EMAP II mediated SphK1/S1P signaling generates cell-type specific response such as macrophage pro-inflammatory activation and SMC proliferation.

Although recent studies target the inhibition of SphK1 as a protective treatment for SphK1/S1P signaling activated inflammatory and proliferative role of altered S1P homeostasis, EMAP II initiated bimodal phosphorylation of SphK1 is believed to be an important upstream regulator of the SphK1/S1P mediated signaling mechanism. Thus, modulating the activity of EMAP II is anticipated to be a more specific and effective therapeutic target in treating several pathophysiological conditions of inflammation and SMC proliferation.

### SUMMARY

The present disclosure relates generally to compositions and methods of modulate the S1P-S1PR signaling pathway. In accordance with one embodiment the activity of EMAP II is reduced by contact with a ligand (e.g., an antibody) that specifically binds to EMAP II. Antibodies to EMAP II have been described in U.S. Pat. No. 5,641,867 and Schluesener et al, GLIA, Vol 20 (4) pages 365-372, December, 1998, the disclosures of which are expressly incorporated herein. However, previously described antibodies to EMAP II, lack a neutralizing function. The antibody disclosed herein targets a very specific region of the EMAP II C-terminus and this antibody actively inhibits EMAP II function.

In an alternative embodiment the activity of EMAP II is reduced by interfering with the cleavage of Aminoacyl tRNA synthetase complex Interacting Multifunctional Protein 1 (AIMPI). In one embodiment the cleavage of AIMPI is inhibited by a caspase peptide-based inhibitor such as an inhibitor having the structure:

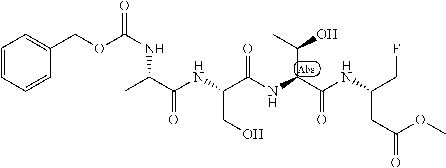 [SMILES: COC(=O)C[C@@H](CF)NC(=O)[C@@H](NC(=O)[C@H](CO)NC(=O)[C@H](C)NC(=O)OCc1ccccc1)[C@@H](C)O]

(Z-ASTD-FMK; EMD Biosciences, Inc.). Z-ASTD-FMK is a cell-permeant and irreversible inhibitor of endothelial monocyte-activated polypeptide II (EMAP II). The peptide sequence of this inhibitor represents the cleavage site of murine pro-EMAP II, whereas the fluoromethylketone residue irreversibly binds to and blocks the central cysteine residue of caspases.

In accordance with one embodiment methods are provided for modulating the S1P-S1PR signaling pathway in a subject by administering an endothelial monocyte activating polypeptide II (EMAP II) inhibitor. Another embodiment of the invention is directed to methods to modulate levels of sphingosine-1-phosphate (S1P) in a subject by administering an endothelial monocyte activating polypeptide II (EMAP II) inhibitor. In one embodiment the EMAP II inhibitor is an antibody that specifically binds to EMAP II.

EMAP II targets an upstream mechanism of PASMC proliferation, that of EGR1. EMAP II regulated activation of EGR1 transcription and translation is shown in FIGS. 10A-10E, 11A-11F and 12A-12F. Therefore, in accordance with one embodiment inhibition of EMAP II activity will lead to inhibition of proposed targets EGR1 and the downstream target of Sphk1. In accordance with one embodiment a method for treating inflammatory disorders and smooth muscle cell proliferation disorders is provided wherein a subject suffering from such a disorder is administered an endothelial monocyte activating polypeptide II (EMAP II) inhibitor. In one embodiment the inflammatory disorder is a chronic respiratory disorder, e.g., bronchopulmonary dysplasia, pulmonary fibrosis, COPD, or other disorder associated with acute lung injury and repair. The smooth muscle cell proliferation disorder may be a vascular disorder such as atherosclerosis, restenosis, or pulmonary hypertension. The EMAP II inhibitor may be an EMAP II signally pathway inhibitor, a blocking antibody, an Erk inhibitor and/or an EGR1 inhibitor. In one embodiment the EMAP II inhibitor is an antibody that specifically binds to EMAP II.

In one embodiment a method of treating bronchopulmonary dysplasia in a subject is provided. In one embodiment the method comprises administering to the subject in need thereof an endothelial monocyte activating polypeptide II (EMAP II) inhibitor. The EMAP II inhibitor may be an EMAP II signally pathway inhibitor, a blocking antibody, an Erk inhibitor and/or an EGR1 inhibitor. In one embodiment the EMAP II inhibitor is an antibody that specifically binds to EMAP II.

### BRIEF DESCRIPTION OF THE DRAWINGS

FIGS. 1A-1E show EMAP II induced STATS activation in macrophages is linked with S1P signaling and ERK activation. (FIG. 1A): Representative western blot probed for pSTAT3 in whole cell lysates of RAW 264.7 cells pretreated with W146 (iSIPR1), JTE013 (iS1PR2), TY52156 (iS1PR3), PF543 (iSphK1), Ruxolitinib (iJak1/2) or vehicle exposed to EMAP II or vehicle for 24 hours. FIG. 1B presents quantitation of pSTAT3/STAT3. FIG. 1C provides data for HEK293-T cells reverse transfected with 200 ng of pGL3-STAT3 and 10 ng of pRL-null vectors, pretreated with W146 (iSIPR1), JTE013 (iS1PR2), TY52156 (iS1PR3), PF543 (iSphK1), Ruxolitinib (iJak1/2) or vehicle exposed to EMAP II or vehicle and luciferase activity measured after 24 hours. FIG. 1D: Representative western blot probed for pSTAT3 in whole cell lysates of RAW 264.7 cells pretreated with PD184161 (iERK) or vehicle exposed to EMAP II or vehicle for 24 hours. FIG. 1E provides quantitation of pSTAT3/STAT3. Results are shown as means±SD. N=4, *p<0.05, **p<0.01, ***p<0.001, ****p<0.0001 using unpaired t-test.

FIGS. 2A-2F show EMAP II induces time dependent bi-modal ERK activation and overexpression of immediate early transcription factor EGR1. FIG. 2A: Representative western blot probed for ERK and EGR1 in whole cell lysates of EMAP II treated RAW 264.7 cells had a time dependent phosphorylation of ERK and an overexpression of EGR1. FIG. 2B provides data showing the quantification of pERK/ERK. FIG. 2C provides data showing the quantification of EGR1/tubulin. FIG. 2D: Representative western blot probed for ERK and EGR1 in whole cell lysates of EMAP II treated hPASMC cells had a time dependent phosphorylation of ERK and an overexpression of EGR1. FIG. 2E provides data showing the quantification of pERK/ERK. (FIG. 2F): quantification of EGR1/tubulin. Results are shown as means±SD. N=3, *p<0.05, **p<0.01, ***p<0.001, ****p<0.0001 using unpaired t-test.

FIGS. 3A-3F show EGR1 upregulation is mediated through EMAP II induced ERK activation. FIG. 3A: Representative western blot probed for EGR1 in whole cell lysates of RAW 264.7 cells pretreated with PD184161 (iERK), Trametinib (iERK1) or vehicle exposed to EMAP II or vehicle 2 hours. FIG. 3B provides data showing the quantitation of EGR1/Tubulin. FIG. 3C: EGR1 mRNA expression levels normalized against Hprt1 in RAW 264.7 cells pretreated with Trametinib (iERK1) or vehicle and followed EMAP II treatment for 45 minutes. FIG. 3D: Representative western blot probed for EGR1 in whole cell lysates of hPASMC cells pretreated with PD184161 (iERK), Trametinib (iERK1) or vehicle exposed to EMAP II or vehicle 1 hour. FIG. 3E provides data showing the quantitation of EGR1/Tubulin. FIG. 3F: EGR1 mRNA expression levels normalized against Gapdh in hPASMC cells pretreated with Trametinib (iERK1) or vehicle and followed EMAP II treatment for 45 minutes. Results are shown as means±SD. N=3, *p<0.05, **p<0.01 using unpaired t-test.

FIGS. 4A-4E show EMAP II induced upregulation of EGR1 expression is nuclear localized. FIG. 4A: Representative western blot probed for EGR1 and pERK in cytoplasmic and nuclear fractions of RAW 264.7 following EMAP II treatment for 0, 1, 2, 3 hours. FIG. 4B shows the quantification of nuclear EGR1/1amin FIG. 4C: Representative western blot probed for EGR1 and pERK in cytoplasmic and nuclear fractions of hPASMC following EMAP II treatment for 0, 20 minutes and 1 and 2 hours. FIG. 4D shows the quantification of nuclear EGR1/1amin FIG. 4E provides the quantification of fluorescence intensity of immunocytochemistry images of EGR1 expression in vehicle (i) or EMAP II (ii) treated hPASMC cells for 1 hour. Results are shown as means±SD. N=3, ns=non-significant, *p<0.05, **p<0.01, **p<0.001 using unpaired t-test.

FIGS. 5A-5K show EMAP II upregulates SphK1 transcription and translation through EGR1. FIG. 5A: Representative western blot probed for SphK1 in whole cell lysates of RAW 264.7 cells following EMAP II treatment for 0, 1, 2, 6 and 24 hours. FIG. 5B shows the quantitation of SphK1/Tubulin. FIG. 5C: SphK1 mRNA expression levels normalized against Hprt1 in RAW 264.7 cells following EMAP II treatment for 0, 1, 3, 6 and 24 hours. FIG. 5D: Representative western blot probed for SphK1 in whole cell lysates of hPASMC cells following treatment with EMAP II 0, 1, 2, 6, 24 hours. FIG. 5E shows the quantitation of SphK1/Tubulin. FIG. 5F: SphK1 mRNA expression levels normalized against Gapdh in hPASMC cells following EMAP II treatment for 0, 1, 3, 6 and 24 hours. FIG. 5G: Representative western blot probed for SphK1 in whole cell lysates of hPASMC cells following siRNA mediated EGR1 silencing and EMAP II treatment for 6 hours. FIG. 5H shows the quantitation of SphK1/Tubulin. FIG. 5I: SphK1 mRNA expression levels normalized against Gapdh in hPASMC cells following siRNA mediated EGR1 silencing and EMAP II treatment for 3 hours. FIG. 5J: Representative western blot probed for EGR1 in whole cell lysates of hPASMC cells following siRNA mediated EGR1 silencing and EMAP II treatment for 6 hours. FIG. 5K quantitation of EGR1/Tubulin. Results are shown as means±SD. N=3, ns=non-significant, *p<0.05, **p<0.01 using unpaired t-test.

FIGS. 6A-6B show EMAP II induces bi-modal phosphorylation of SphK1. FIG. 6A: Representative western blot probed for pSphK1 in whole cell lysates of hPASMC cells following EMAP II treatment for 0, 1, 2, 6 and 24 hours. FIG. 6B shows quantitation of pSphK1/Tubulin.

FIGS. 7A-7G show EMAP II modulates S1P levels. FIG. 7A: Cellular C18-SIP and sphingosine levels, FIG. 7B heat map representation of cellular C18-S1P and sphingosine levels and FIG. 7C cellular ceramide levels (C16-Cer, C18-Cer, C20-Cer, C22-Cer and C24-Cer) normalized against cell phosphates in RAW 264.7 cells following EMAP II treatment for 0, 1, 2, 6 and 24 hours. FIG. 7D: Cellular C18-SIP and sphingosine levels, FIG. 7E heat map representation of cellular C18-S1P and sphingosine levels and FIG. 7F: cellular ceramide levels (C16-Cer, C18-Cer, C20-Cer, C22-Cer and C24-Cer) normalized against cell phosphates in hPASMC cells following EMAP II treatment for 0, 1, 2, 6 and 24 hours. FIG. 7G: Extracellular C18-SIP in 1 mL of hPASMC growth media at o and 1 hour of EMAP II treatment. Results are shown as means±SD. N=3 to 5.

FIGS. 8A-8E show the downstream effects of EMAP II/SIP signaling are cell type specific. FIG. 8A: Representative western blot probed for Cyclin D1 in whole cell lysates of hPASMC cells pretreated with PD184161 (iERK), EMAP II neutralizing antibody (Anti-EMAP II) or vehicle following EMAP II or vehicle treatment for 48 hours. FIG. 8B shows the quantitation of Cyclin D1/Tubulin. FIG. 8C: Cell proliferation rate in hPASMC cells pretreated with PF543 (iSphK1) or vehicle following EMAP II treatment for 48 hours. FIG. 8D: Il6 mRNA expression levels normalized against Gapdh in hPASMC cells following EMAP II treatment for 0, 1, 3, 6 and 24 hours. FIG. 8E: Tnf mRNA expression levels normalized against Hprt1 in RAW 264.7 cells following EMAP II treatment for 0, 1, 3, 6 and 24 hours. Results are shown as means±SD. N=3, ns=non-significant, *p<0.05, **p<0.01 using unpaired t-test.

FIG. 9A-9G. EMAP II modulates in vitro and in vivo S1P levels. (FIG. 9A): ELISA-cellular C18-S1P levels normalized against 1 μg of protein, (FIG. 9B): HPLC/MS/MS-cellular C18-S1P and sphingosine levels and (FIG. 9C): HPLC/MS/MS-cellular ceramide levels (C16-Cer, C18-Cer, C20-Cer, C22-Cer and C24-Cer) normalized against cell phosphates in hPASMC cells following EMAP II treatment for 0, 1, 2, 6 and 24 hours. (FIG. 9D): HPLC/MS/MS-extracellular C18-SIP in 1 mL of hPASMC growth media at 0 and 1 hour of EMAP II treatment. (FIG. 9E): HPLC/MS/MS-cellular C18-S1P and sphingosine levels, (FIG. 9F): HPLC/MS/MS-cellular ceramide levels (C16-Cer, C18-Cer, C20-Cer, C22-Cer and C24-Cer) normalized against cell phosphates in RAW 264.7 cells following EMAP II treatment for 0, 1, 2, 6 and 24 hours. (FIG. 9G): HPLC/MS/MS tissue C18-S1P levels normalized against 1 mg of protein in postnatal day 15 mouse lung tissues following EMAP II treatment on postnatal days 3-15. Results are shown as means±SEM. n=3, *p<0.05.

FIGS. 10A-10E EMAP II induced STATS activation in macrophages is linked with S1P signaling and ERK activation. (FIG. 10A): Representative western blot probed for pSTAT3 in whole cell lysates of RAW 264.7 cells pretreated with W146 (iSIPR1), JTE013 (iS1PR2), TY52156 (iS1PR3), PF543 (iSPHK1), Ruxolitinib (iJak1/2) or vehicle exposed to EMAP II or vehicle for 24 hours. (FIG. 10B): quantitation of pSTAT3/STAT3. (FIG. 10C): HEK293-T cells were reverse transfected with 200 ng of pGL3-STAT3 and 10 ng of pRL-null vectors, pretreated with W146 (iSIPR1), JTE013 (iS1PR2), TY52156 (iS1PR3), PF543 (iSPHK1), Ruxolitinib (iJak1/2) or vehicle exposed to EMAP II or vehicle and luciferase activity measured after 24 hours. (FIG. 10B) and (FIG. 10C): Following one-way ANOVA, Turkey's honestly significant difference test, *p<0.05, **p<0.01, ****p<0.0001. (FIG. 10D): Representative western blot probed for pSTAT3 in whole cell lysates of RAW 264.7 cells pretreated with PD184161 (iERK) or vehicle exposed to EMAP II or vehicle for 24 hours. (FIG. 10E): quantitation of pSTAT3/STAT3. Results are shown as means±SEM. n=3, *p<0.05, **p<0.01, ***p<0.001, ****p<0.0001.

FIGS. 11A-11F EMAP II induces time dependent bi-modal ERK activation and overexpression of immediate early transcription factor EGR1. (FIG. 11A): Representative western blot probed for ERK and EGR1 in whole cell lysates of EMAP II treated RAW 264.7 cells had a time dependent phosphorylation of ERK and an overexpression of EGR1. (FIG. 11B): quantification of pERK/ERK. (FIG. 11C): quantification of EGR1/tubulin. (FIG. 11D): Representative western blot probed for ERK and EGR1 in whole cell lysates of EMAP II treated hPASMC cells had a time dependent phosphorylation of ERK and an overexpression of EGR1. (FIG. 11E): quantification of pERK/ERK. (FIG. 11F): quantification of EGR1/tubulin. Results are shown as means±SEM. n=3, *p<0.05, **p<0.01, ***p<0.001, ****p<0.0001.

FIGS. 12A-12F EGR1 upregulation is mediated through EMAP II induced ERK activation. (FIG. 12A): Representative western blot probed for EGR1 in whole cell lysates of RAW 264.7 cells pretreated with PD184161 (iERK), Trametinib (iERK1) or vehicle exposed to EMAP II or vehicle for 2 hours (FIG. 12B): quantitation of EGR1/Tubulin. (FIG. 12C): EGR1 expression levels normalized against Hprt1 in RAW 264.7 cells pretreated with Trametinib (iERK1) or vehicle and followed EMAP II treatment for 45 minutes. (FIG. 12D): Representative western blot probed for EGR1 in whole cell lysates of hPASMC cells pretreated with PD184161 (iERK), Trametinib (iERK1) or vehicle exposed to EMAP II or vehicle for 1 hour. (FIG. 12E): quantitation of EGR1/Tubulin. (FIG. 12F): EGR1 expression levels normalized against Gapdh in hPASMC cells pretreated with Trametinib (iERK1) or vehicle and followed EMAP II treatment for 45 minutes. Results are shown as means±SEM. n=3, *p<0.05, **p<0.01.

FIGS. 13A-13E EMAP II induced upregulation of EGR1 expression is nuclear localized. (FIG. 13A): Representative western blot probed for EGR1 and pERK in cytoplasmic and nuclear fractions of RAW 264.7 following EMAP II treatment for 0, 1, 2, 3 hours. (FIG. 13B): quantification of nuclear EGR1/1amin. (FIG. 13C): Representative western blot probed for EGR1 and pERK in cytoplasmic and nuclear fractions of hPASMC following EMAP II treatment for 0, 20 minutes and 1 and 2 hours. (FIG. 13D): quantification of nuclear EGR1/1amin (FIG. 13F): quantification of fluorescence intensity of EGR1 from immunocytochemistry images of EGR1 expression in vehicle (i) or EMAP II (ii) treated hPASMC cells for 1 hour. Results are shown as means±SEM. n=3, ns=non-significant, *p<0.05, **p<0.01, ***p<0.001,

FIGS. 14A-14K EMAP II upregulates SPHK1 transcription and translation through EGR1. (FIG. 14A): Representative western blot probed for SPHK1 in whole cell lysates of RAW 264.7 cells following EMAP II treatment for 0, 1, 2, 6 and 24 hours. (FIG. 14B): quantitation of SPHK1/Tubulin. (FIG. 14C): SPHK1 expression levels normalized against Hprt1 in RAW 264.7 cells following EMAP II treatment for 0, 1, 3, 6 and 24 hours. (FIG. 14D): Representative western blot probed for SPHK1 in whole cell lysates of hPASMC cells following treatment with EMAP II 0, 1, 2, 6, 24 hours. (FIG. 14E): quantitation of SPHK1/Tubulin. FIG. 14F SPHK1 expression levels normalized against Gapdh in hPASMC cells following EMAP II treatment for 0, 1, 3, 6 and 24 hours. (FIG. 14G): Representative western blot probed for SPHK1 in whole cell lysates of hPASMC cells following siRNA mediated EGR1 silencing and EMAP II treatment for 6 hours. (FIG. 14H): quantitation of SPHK1/Tubulin. (FIG. 14I): SPHK1 expression levels normalized against Gapdh in hPASMC cells following siRNA mediated EGR1 silencing and EMAP II treatment for 3 hours. (FIG. 14J): Representative western blot probed for EGR1 in whole cell lysates of hPASMC cells following siRNA mediated EGR1 silencing and EMAP II treatment for 6 hours. (FIG. 14K): quantitation of EGR1/Tubulin. Results are shown as means±SEM. n=3, ns=non-significant, *p<0.05, **p<0.01.

FIGS. 15A-15B EMAP II induces bi-modal phosphorylation and plasma membrane localization of SPHK1. (FIG. 15A): Representative western blot probed for pSPHK1 in whole cell lysates of hPASMC cells following EMAP II treatment for 0, 1, 2, 6 and 24 hours. (FIG. 15B): quantitation of pSPHK1/Tubulin.

FIGS. 16A-16E The downstream effects of EMAP II/SIP signaling are cell type specific. (FIG. 16A): Representative western blot probed for Cyclin D1 in whole cell lysates of hPASMC cells pretreated with PD184161 (iERK), EMAP II neutralizing antibody (Anti-EMAP II) or vehicle following EMAP II or vehicle treatment for 48 hours. (FIG. 16B): quantitation of Cyclin D1/Tubulin. (FIG. 16C): Cell proliferation rate in hPASMC cells pretreated with PF543 (iSPHK1) or vehicle following EMAP II treatment for 48 hours. (FIG. 16D): Il6 expression levels normalized against Gapdh in hPASMC cells following EMAP II treatment for 0, 1, 3, 6 and 24 hours. (FIG. 16E): Tnf expression levels normalized against Hprt1 in RAW 264.7 cells following EMAP II treatment for 0, 1, 3, 6 and 24 hours. Results are shown as means±SEM. n=3 or 4, ns=non-significant, *p<0.05, **p<0.01.

FIG. 17 is a schematic representation of the potential mechanism of EMAP II/SIP signaling and modulation of S1P homeostasis. EMAP II stimulation activates a common signaling pathway where ERK activation induces bimodal phosphorylation of SPHK1 and EGR1 overexpression. EGR1 overexpression upregulates SPHK1 transcription and translation. Increased SPHK1 and phosphorylated SPHK1 results in augmented S1P levels which generates cell type-specific downstream effects such as pro-inflammatory signals in macrophages and pro-proliferative signals in SMCs.

### DETAILED DESCRIPTION

### Abbreviations

SMC Smooth Muscle Cell
miRNA microRNA
siRNA small interference RNA
AIMPI Aminoacyl tRNA synthetase complex Interacting Multifunctional Protein 1 (also known as p43/proEMAP II)
EMAP II Endothelial Monocyte Activating Polypeptide II
STAT signal transducer and activators of transcription
S1P Sphingosine-1-Phosphate
S1PR S1P Receptor
ERK Extracellular Signal-regulated Kinase
SphK Sphingosine kinase
EGR1 Early Growth Response 1
iERK PD184161
iERK1 Trametinib
PH pulmonary hypertension
PASMC pulmonary artery smooth muscle proliferation

### Definitions

Unless defined otherwise, all technical and scientific terms used herein have the same meaning as commonly understood by one of ordinary skill in the art to which the disclosure belongs. In describing and claiming the invention, the following terminology will be used in accordance with the definitions set forth below.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “treating” includes prophylaxis of the specific disorder or condition, or alleviation of the symptoms associated with a specific disorder or condition and/or preventing or eliminating said symptoms.

The term, “parenteral” means not through the alimentary canal but by some other route such as subcutaneous, intramuscular, intraspinal, or intravenous.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition. The term “purified RNA” is used herein to describe an RNA sequence which has been separated from other compounds including, but not limited to polypeptides, lipids and carbohydrates.

The term “isolated” requires that the referenced material be removed from its original environment (e.g., the natural environment if it is naturally occurring). For example, a naturally-occurring nucleic acid present in a living animal is not isolated, but the same nucleic acid, separated from some or all of the coexisting materials in the natural system, is isolated.

As used herein, the expression “solid support” means any solid surface to which an analyte-binding molecule (e.g., antibody or antigenically reactive fragment thereof) can be attached such that the analyte-binding molecule cannot break free from the solid support in a liquid medium. A solid support can easily be separated from a liquid which the solid support contacts. In varying embodiments, the solid support can be, for example, plastic, derivatized plastic, magnetic or non-magnetic metal, glass or silicon. Representative examples of solid supports, include without limitation, electrodes, test tubes, beads, microparticles, nanoparticles, wells of micro- or multi-well plates, gels, colloids, biological cells, sheet, chip, and other configurations known to those of ordinary skill in the art. Moreover, optionally the solid support provides a means of recovery of the analyte-binding protein—i.e., means of release or detachment of the analyte-binding molecule from the surface under controlled conditions distinct from those in which the assay is conducted. For example, the analyte-binding molecule may be attached to the solid support by means of a cleavable linker.

As used herein an “antibody” includes monoclonal antibodies, multispecific antibodies, bifunctional antibodies, recombinant antibodies, chimeric antibodies, single-chain Fvs (“scFv”), single chain antibodies, single domain antibodies, Fab fragments, F(ab′) fragments, F(ab′)2 fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25(11): 1290-1297 (2007), and International Patent Application Publication No. WO 2001/058956, the contents of each of which are herein incorporated by reference), and functionally active epitope-binding fragments of any of the above. The term “bifunctional antibody,” as used herein, refers to an antibody that comprises a first arm having a specificity for one antigenic site and a second arm having a specificity for a different antigenic site, i.e., the bifunctional antibodies have a dual specificity.

An “antibody fragment” as used herein refers to a portion of an intact antibody comprising the antigen-binding site or variable region. The portion does not include the constant heavy chain domains (i.e., CH2, CH3 or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

As used herein the term “patient” without further designation is intended to encompass any warm blooded vertebrate domesticated animal (including for example, but not limited to livestock, horses, mice, cats, dogs and other pets) and humans.

As used herein, “a subject in need thereof” refers to a subject having, susceptible to or at risk of a specified disease, disorder, or condition. More particularly, in the present disclosure the methods of screening biomarkers are to be used with a subset of subjects who have, are susceptible to or are at an elevated risk for experiencing hyperglycemia and type 1 diabetes. Such subjects can be susceptible to or at elevated risk for hyperglycemia and type 1 diabetes due to family history, age, environment, and/or lifestyle.

As used herein, “susceptible” and “at risk” refer to having little resistance to a certain disease, disorder or condition, including being genetically predisposed, having a family history of, and/or having symptoms of the disease, disorder or condition.

### EMBODIMENTS

Following tissue injury, bioactive sphingolipids, including S1P, function as secreted signaling mediators involved in key steps of the stress response regulating local inflammation, cell survival, perivascular smooth muscle proliferation, and angiogenesis. Elevated S1P levels in tracheal fluid isolated from premature infants that developed BPD, as well as in genome wide analysis of a hyperoxia mouse model of BPD, further emphasize the impact of this lipid to the pathogenesis of BPD. Following activation of upstream tyrosine kinase receptors, a bi-modal pattern of S1P phosphorylation is initiated by early phosphorylation of existing SphK1 and delayed phosphorylation of transcriptionally regulated SphK1. Generation of S1P at the cell membrane by SphK1 phosphorylation of sphingosine allows S1P transporter secretion where it acts as a ligand for the ubiquitously expressed G protein-coupled S1P receptors (S1PR) found on every cell type identified, leading to a plethora of cell specific responses including inflammation, differentiation, migration, and cell proliferation. Consequently, regulating the phosphorylation of sphingosine to S1P is vital in controlling the S1P/S1PR signaling cascade. Although SphK phosphorylation of sphingosine is identified as a decisive toggle point in the activation of the sphingosine SphK S1P S1PR signaling axis providing a distinct regulatory site, the inflammatory consequences of controlling one aspect of the bi-modal pSphK peak, post-translation versus transcription, is unknown. In one aspect, the present disclosure is directed to a method of regulating an S1P mediated response in a subject.

Endothelial Monocyte Activating Polypeptide (EMAP) II, a potent pro-inflammatory cytokine whose sustained neonatal expression invokes a BPD mimic of alveolar simplification and pulmonary hypertension is known to signal through tyrosine kinases. We have identified an important upstream signaling link between EMAP II and S1P that is conserved in inflammatory and smooth muscle cells (SMC). As disclosed herein: 1) EMAP II activation of the ERK (Extracellular Signal-regulated Kinase) signaling pathway regulates early and late SphK1 phosphorylation, 2) EMAP II regulates SphK1 transcription through the transcriptional regulator of differentiation, inflammation, and mitogenesis protein coding gene EGR1 (Early Growth Response 1), 3) Blockade of upstream ERK signal transduction cascade prevents EMAP II mediated bi-modal SphK1 phosphorylation, and 4) EMAP II increases S1P. Downstream, EMAP II induces cell and S1PR specific S1P signaling as inflammatory signaling is activated in macrophages while proliferation is targeted in SMC. The present disclosure is directed to disruption of the conserved upstream activation of SphK to prevent the cell specific inflammatory and proliferative consequences of bioactive sphingolipid S1P signaling pathway.

Regulating inflammation in prematurely born infants could have therapeutic implications in pulmonary development. BPD, a chronic lung disorder common among children born prematurely, is characterized by alveolar simplification, inflammation, restrictive lung physiology, as well as pervasive vascular defects. Despite advances in clinical ventilator management, the introduction of surfactant, and antenatal glucocorticoids, there is a marked lack of adjunctive therapies. Several investigators have identified a direct link between inflammation, distal alveolar structure, and lung dysplasia. In extremely premature infants born as early as 23-28 weeks of age within the particularly critical mid-canalicular to saccular stages of development designed for the formation of a cohesive and functional air-exchanging alveolar-capillary interface, infants are exposed to external environmental and respiratory influences. During this vulnerable stage, in part due to supportive measures, the lungs' innate immune response is activated. Although less is known regarding the inflammatory response of the immature neonatal lung, recent studies suggest that local cellular response to hyperoxia contributes to the inflammatory state. Prior to influx of inflammatory cells, oxidative and cellular damage triggers early response chemokines such as the ‘Find me’ mediators EMAP II and bioactive lipid SIP that recruit macrophages to the region of injury and amplifies lung damage through induction of cytokines such as IL1, IL6 and Tnf. However, blockade of macrophage-secreted cytokines IL-1 and TNF showed little improvement in mouse models, suggesting that other strategies are required.

Bioactive sphingolipids have gained increasing consideration as important regulatory signaling molecules within the pathophysiologic response to inflammation, respiratory distress, cardiovascular disease, and cancer. Although these pleotropic bioactive lipid mediators are protective in sepsis, they are deleterious in other disease processes such as hyperoxic lung injury and pulmonary hypertension. As the predominate lipid constituent of all cellular membranes, sphingolipids are defined by their eighteen carbon amino-alcohol backbone that is synthesized in the endoplasmic reticulum where sphingosine serves as one of the simplest backbones that can achieve further complexity through phosphorylation. Catalyzed by two isoforms of sphingosine kinase (SphK1 and SphK2), S1P is phosphorylated from sphingosine and can be reversibly phosphorylated by sphingosine phosphatases and irreversibly by SIP lyase to maintain the S1P levels. Once phosphorylated, S1P will act as either an intracellular secondary messenger, or be transported extracellular via the transporter Spns2 (spinster homologue 2). Extracellularly, an S1P gradient is formed that actively binds to one of five G protein-coupled receptors S1PR1-5. This binding results in a unique signaling axis as S1P-S1PR regulates itself in an inside-out manner while controlling a broad host of downstream targets depending on cell type and the S1P receptor. Elevated S1P levels promote S1PR internalization followed by their recycling back to the cell surface within several hours providing a feedback system that takes advantage of the S1P gradient. Upon binding of S1P to its receptor, cell signaling responses in part through activation of the JAK/STAT signaling pathway (Janus tyrosine kinase/signal transducers and activators of transcription) range from immune cell trafficking and migration, cellular proliferation and survival, endothelial cell permeability, actin dynamics to anti-migration and inflammation. Recent studies associate elevated tracheal fluid S1P with the formation of BPD, where premature infants with the chronic lung disorder BPD have elevated S1P levels at postnatal days 1-2. Similarly, in a neonatal murine hyperoxia model of BPD, expression of the S1P phosphorylating kinase SphK1 was elevated in lung tissue, while its ablation significantly suppressed pro-inflammatory and apoptotic genes while improving alveolarization associated with hyperoxia induced BPD. In addition to stimulating inflammation, SphK1 mediated S1P promotes pulmonary smooth muscle cell proliferation contributing to vascular remodeling/vasoconstriction in both rodent and human pulmonary arterial smooth muscle cells. These findings strongly support a pro-inflammatory and pulmonary arterial hypertension role for S1P in lung disease of prematurity and suggest that regulation of sphingosine phosphorylation by SphK1 could represent an effective target to mediate the inflammation and pulmonary hypertension of BPD.

Diverse spectrums of cell specific responses are attributed to S1P due to the ubiquitous nature of its five different G protein-coupled receptors and their unique and variable cell specific expression. Therefore, an effective method of mediating SIP downstream signaling could be found upstream through regulation of SphK phosphorylation. As SphK is the phospholipid enzyme that regulates sphingosine phosphorylation to SIP, its activity largely regulates cellular and extracellular SIP levels. Although SphK isoenzymes (SphK1 and SphK2) and isoforms (hSphK1a:42.5 kDa, hSphK1b:51.51(Da, HSphK1c:43.0 kDa, hSphK2a, and hSphk2b) demonstrate some redundancy in function, Sphk1 isoenzyme specific expression has been linked to distinct cellular responses in disease such as hyperoxic induced lung disease of prematurity and treatment resistance in cancer. Identified as a housekeeping enzyme to regulate sphingosine, ceramide, and S1P levels, SphK1 levels are maintained at a low basal activity that can rapidly be activated through direct phosphorylation of SphK1 by phosphorylated ERK1/2 (Extracellular signal regulated kinases 1 and 2) at Ser225 resulting in a 14-fold increase in SphK1 catalytic activity. Fundamental to SphK1 activity is its cellular localization. Normally residing in the cytoplasm, upon Ser225 phosphorylation by ERK1/2, SphK1 translocates to the plasma membrane where binding via a CaM-related calcium and integrin-binding protein 1 (CIB1) provides a plasma membrane docking location via myristoylation where its retention facilitates sphingosine phosphorylation. Replenishment of the SphK1 pool is initiated through transcriptional regulation. Encoded on chromosome 17, Sphk1 protein is found predominately in the cytosol and plasma membrane, mediated by transcription factors regulation of SphK1 transcription including EGR1 (Early Growth Response 1), E2F transcription factor 1,4 and TALI erythroid differentiation factor. In support of EGR1 as a transcriptional regulator of SphK1 in BPD and pulmonary hypertension disease processes, EGR1 is identified as one of the early biomarkers of lung injury in preterm lambs where mRNA levels were increased 120-fold within 30 minutes of injury and is associated with increased lung injury. Consistent with these findings, EGR1 suppression rescued airway hyper-responsiveness, lung remodeling, and vascular muscularization. Importantly, ERK1/2 phosphorylation has a second influence on SphK1. In addition to ERK1/2 mediating cytoplasmic SphK1 phosphorylation, it also mediates EGR1 transcription. Following ERK1/2 phosphorylation it's translocation into the nucleus induces EGR1 transcription. In addition to increased lung injury, a xanthine oxidase mediated increase of EGR1 mRNA and protein through an ERK phosphorylation mediated process contributes to the pulmonary artery smooth muscle proliferation in newborn calves consistent with the development of pulmonary hypertension supporting a role for ERK1/2 mediated ERG1 transcriptional regulation in pulmonary vascular remodeling. Therefore phosphorylation of ERK1/2 is a key regulator of both SphK1 phosphorylation and transcriptional regulation thus providing a critical target point in regulation of downstream sphingosine phosphorylation. What is unclear is the contribution of bi-modal early versus late SphK1 phosphorylation to premature infants pulmonary inflammatory state.

The pervasively expressed 34-kDa intracellular protein Aminoacyl tRNA Synthetase Complex Interacting Multifunctional Protein 1 (AIMP1, also known as SCYE-1, p43, and EMAP II) is one of three scaffold proteins for the multi-synthetase complex (MSC) that mediate protein translation. Prevalent in early lung development, its expression is inversely correlated to periods of vascularization. On the cell surface, it undergoes proteolytic cleavage to generate an extracellular 22-kDa C-terminal peptide EMAP II (Endothelial Monocyte Activating Polypeptide II) that functions as a potent pro-inflammatory cytokine known to chemoattract macrophages and induce TNFα secretion. As a promiscuous signaling cytokine, EMAP IIs' predominate targets are tyrosine kinase receptors, where it functions as an endothelial cell anti-angiogenic protein through an 51 integrin inhibition of endothelial cell adhesion to fibronectin, blockade of fibronectin matrix assembly, and interference with VEGF (vascular endothelial growth factor) pro-angiogenic signaling. Sustained elevation of EMAP II during the formative saccular stage, is associated with an inflammatory state and arrested alveolar and characteristics consistent with pulmonary hypertension in the developing lung while its neutralization in a hyperoxia model of premature lung disease to suppress pro-inflammatory genes and chemotactic genes, while providing protection from a BPD phenotype.

Despite a solid understanding of EMAP IIs' pro-inflammatory impact on lung development, little is understood regarding the mechanisms that regulate its inflammatory response. The data presented herein indicates that EMAP II induces a JAK dependent biphasic tyrosine phosphorylation (p) of STAT3 (Y705) in partially activated thioglycollate-elicited macrophages (TEPM) partially activated peritoneal macrophages, stimulates pSTAT3 nuclear translocation and increases STAT3 mediated transcriptional target genes, many of which were pro-inflammatory. Supportive of EMAP II induced pSTAT3 mediated gene transcription, cells transfected with a luminescence STAT3 transcriptional reporter plasmid had significantly increased luminescence while inhibition of STAT3 phosphorylation ablates transcription of many of the STAT3 mediated target genes. Of particular interest, was the observation that EMAP II induction of STAT3 mediated gene transcripts paralleled several of the known S1P mediated gene transcripts. In line with this observation, addition of S1P receptor inhibitors blocked EMAP II induction of pSTAT3 as well as prevented EMAP II mediated luminescence of STAT3 transcriptional reporter plasmid suggesting that EMAP IIs' pro-inflammatory effect was upstream of S1PR. Exemplary inhibitors for use in accordance with the present disclosure are provided in Table 1:

TABLE 1

| Inhibitor for | Structure | Vendor |
|---|---|---|
| S1PR1 (W146) | 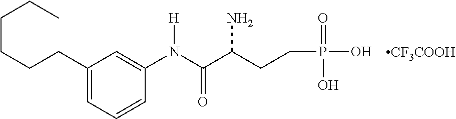 [SMILES: CCCCCCc1cccc(NC(=O)[C@H](N)CCP(=O)(O)O)c1 ; "·CF3COOH"] | Cayman |
| S1PR2 (JTE 013) | 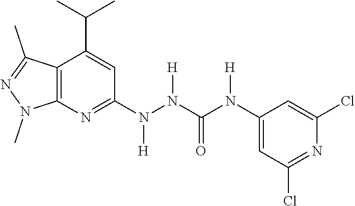 [SMILES: Cc1nn(C)c2nc(NNC(=O)Nc3cc(Cl)nc(Cl)c3)cc(C(C)C)c12] | Cayman |
| S1PR3 (TY 52156) | 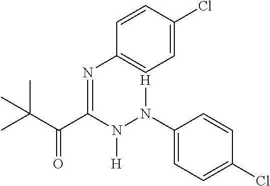 [SMILES: CC(C)(C)C(=O)/C(=N/c1ccc(Cl)cc1)NNc1ccc(Cl)cc1] | Cayman |
| SPHK1 (PF 543) | 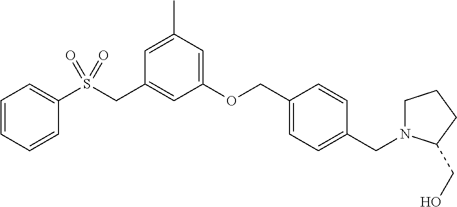 [SMILES: Cc1cc(CS(=O)(=O)c2ccccc2)cc(OCc2ccc(CN3CCC[C@@H]3CO)cc2)c1] | Cayman |
| ERK phosphorylation (PD 184161) | 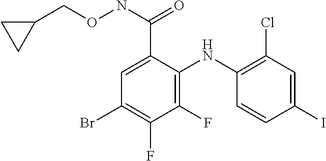 [SMILES: O=C(NOCC1CC1)c1cc(Br)c(F)c(F)c1Nc1ccc(I)cc1Cl] | Santa Cruz |
| ERK phosphorylation (Trametinib) | 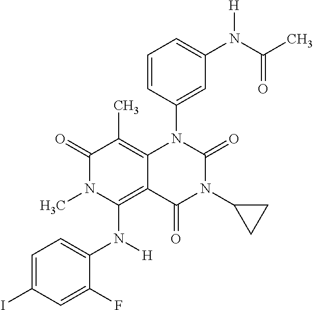 [SMILES: CC(=O)Nc1cccc(-n2c(=O)n(C3CC3)c(=O)c3c(Nc4ccc(I)cc4F)n(C)c(=O)c(C)c32)c1] | LC laboratories |
| Jak1/2 (Ruxolitinib) | 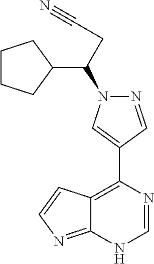 | LC laboratories |

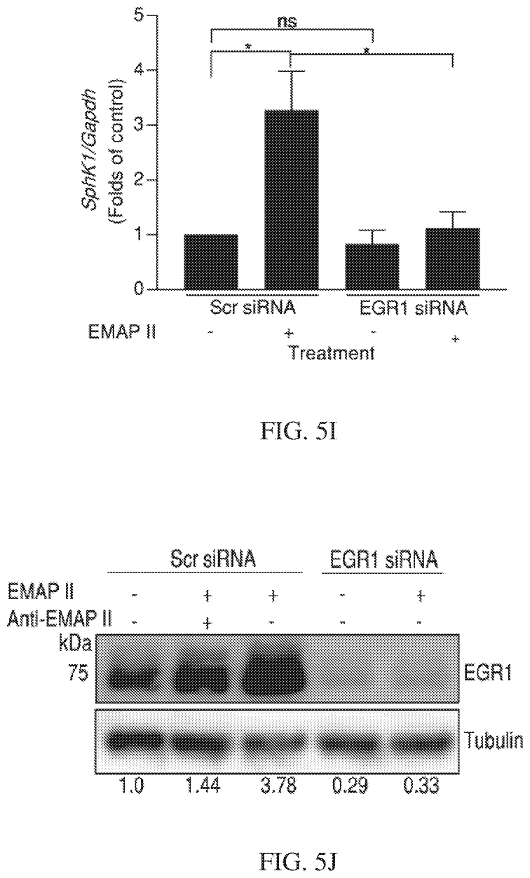

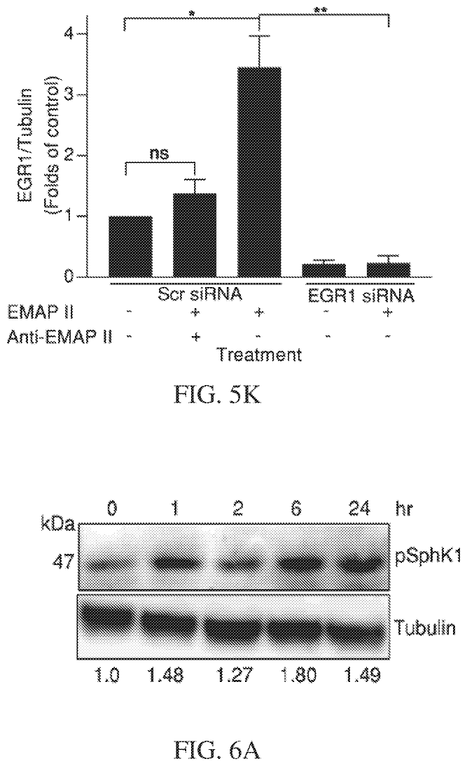

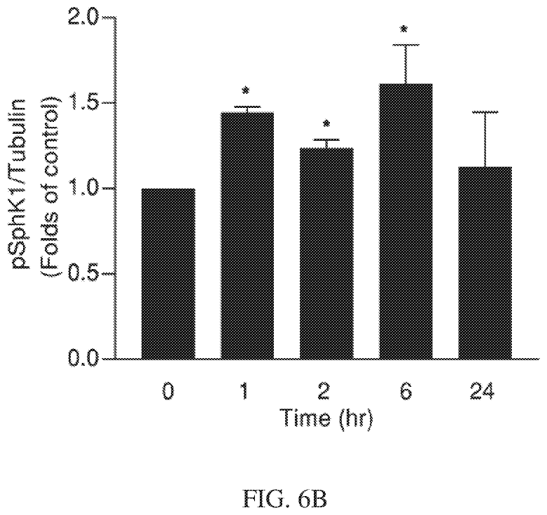

Chemical blockade of SphK using PF543 inhibited pSTAT3. As EMAP II is a promiscuous mediator of tyrosine kinase signaling, we explored the impact of EMAP II on a common tyrosine kinase-signaling factor, ERK1/2. Our data indicates that EMAP II induced phosphorylation of ERK1/2 in a biphasic manner in macrophages. EMAP II induction of pERK was not limited to macrophages as hPASMC (human pulmonary artery smooth muscle cells) also demonstrated similar ERK signaling in response to EMAP II and phosphorylation of SphK in a biphasic manner (FIGS. 6A-6E).

As pERK1/2 mediates SphK1 phosphorylation and transcription, we explored the impact of EMAP II on pERK nuclear translocation and SphK1 transcription. Our data indicates that EMAP II induces nuclear translocation of pERK, an increase nuclear ERG1 protein expression and SphK1 transcription. Importantly, although EMAP II activation of pSphK1 and the resulting increase in S1P release are ubiquitous to two different cell populations, macrophages and pulmonary smooth muscle cells, cellular response to extracellular S1P is cell and S1PR specific as macrophages response to EMAP II was the transcription and secretion of pro-inflammatory cytokines while pulmonary smooth muscle cells experience a marked increase in the proliferation promoting Cyclin D1. These findings suggest that EMAP II regulation of sphingosine SphK S1P is a conserved process through multiple cell types, while cell specific responses driven by S1P S1PR signaling is dependent on cell type and S1PR receptor. Taken together, we hypothesize that EMAP II is a mediator of the sphingosine SphK S1P S1PR signaling axis through regulation of pERK1/2/ERG1 mediated SphK transcription and phosphorylation resulting in release of S1P to bind to cell specific S1PR receptors.

Minimizing the pro-inflammatory environment found in the developing lungs of premature infants is essential to supporting formation of a cohesive and functional air-exchanging alveolar-capillary interface. While studies that focus on the reduction of environmental stressors seems a promising approach, these studies fail to limit inflammation and restore normal lung development in part due to the diverse receptor numbers and induction of transcription factors stimulated by the inflammatory process. While the cell specific consequences of activation of bioactive lipids are known, knowledge of the impact that prolonged exposure to bioactive lipids has in the multi-cellular developing lung is limited. We believe that a multipronged approach aimed at limiting the downstream diverse and cell specific impact of S1P in BPD, is to target the conserved upstream activation pathway. EMAP II, through a signaling mechanism capable of broadly signaling through tyrosine kinases, initiates a bi-modal activation process of the bioactive lipid S1P through mediation of SphK1 phosphorylation and transcription. This conserved upstream acute response system, initiates multiple downstream responses that are largely dependent on cell specific S1PR signaling.

Pulmonary Arterial Hypertension (PAH) is a progressive, incurable and fatal cardiopulmonary vascular disease. Despite the recent advances in etiological and treatment landscape of PAH, it still remains difficult to be diagnosed, treated and managed. PAH can be idiopathic or associated with other diseases resembling idiopathic PAH suggesting that there are similarities in mechanism of disease progression. Pathogenesis of PAH is multifactorial with hypoxia, viral infections, congenital heart disease, and other physiological/exogenous being triggers of PAH. Untreated PAH leads to right ventricular hypertrophy, overload and heart failure. Pulmonary vascular remodeling in concert with vascular proliferation/fibrosis and vessel obstruction are cardinal histopathological marks in PAH. Recent patients and animal studies of PAH revealed that inflammation and angiogenesis are alluring contributors to vessel wall remodeling and cellular hyperproliferation, hallmarks of severe PAH.

Progression of PAH is allied with multiple cell types in the pulmonary arterial wall and pulmonary arterial circulation that contribute to the injury response. Pulmonary arterial smooth muscle cells (PASMC) and inflammatory cells are two major cellular populations that contribute to the pathogenesis of PAH. Microvascular and immuno-modulators such as Sphingosine 1-phosphate (SIP), induced upon injurious stimuli, direct vascular remodeling through a plethora of pathologic cellular processes in different cell types. Hyper-proliferation of PASMC is a vital characteristic in PAH pathogenesis leading to prominent intimal thickening and muscularization of the small arterioles. Recent studies have provided a glimpse at SIP signaling as a regulator of PASMC proliferation while S1P elevated levels in human PAH lungs, suggests that modulation of S1P levels and understanding the signaling cascade of S1P in the development of PAH would unravel therapeutic targets to treat PAH.

Phosphorylation of sphingosine by sphingosine kinases (SPHK1 and SPHK2) generates the bioactive sphingolipid SIP, a key mediator of various biological processes such as cell proliferation and growth, apoptosis, inflammation, angiogenesis and vascular integrity. S1P is present at low intracellular concentrations under tight spatial-temporal control. Upon tissue injury or stress, intracellular levels of prototypical signal molecule, S1P is induced which in turn can function as an intracellular secondary messenger or be transported out of the cells and act as an extracellular signaling molecule by binding to one of five cell-specific G-protein coupled receptors, S1P receptor 1 (S1PR1)-S1P receptor 5 (S1PR5) in autocrine or paracrine manner ‘Inside-out’ relocation of S1P can activate several signaling pathways including Janus tyrosine kinase/signal transducer and activators of transcription (JAK/STAT) and Extracellular Regulated Kinases (ERK). Importantly, during tissue injury and homeostasis early tissue injury response signaling molecules such as S1P recruit phagocytes and induce secretion of proinflammatory cytokines such as IL-1β, IL6 and TNFα 15,16.

As disclosed herein another key microvascular/immune-early response mediator, Endothelial Monocyte Activating Polypeptide II (EMAP II) is also involved in modulating S1P homeostasis and key intermediates in the SPHK1/S1P signaling pathway. As disclosed herein a link between S1P and EMAP II has been identified as EMAP II mediates a distinct inflammatory gene profile in recruited macrophages through a JAK/STAT signaling pathway similar to S1P signaling axis.

As disclosed herein EMAP II is a strategic early regulator of SPHK1 mediated S1P expression where it signals in both macrophages and SMCs through ERK1/2 and transcriptional regulator Early growth response 1 (EGR1). Furthermore, EMAP II mediated SPHK1/S1P signaling generates cell-type specific response such as macrophage pro-inflammatory activation and PASMC proliferation. Although recent studies target the inhibition of SPHK1 as a protective treatment for SPHK1/S1P signaling activated inflammatory and proliferative role of altered S1P homeostasis, the results described herein suggest that EMAP II initiated bimodal phosphorylation of SPHK1 is an important upstream regulator of the SPHK1/S1P mediated signaling mechanism. Thus providing a specific and effective therapeutic target for treating PAH and several pathophysiological conditions of inflammation and PASMC proliferation.

In accordance with one embodiment cell specific inflammatory and proliferative consequences of bioactive sphingolipid S1P signaling pathway are reduced by administering to the subject in need thereof an endothelial monocyte activating polypeptide II (EMAP II) inhibitor. In one embodiment the EMAP II inhibitor comprises an EMAP II signaling pathway inhibitor, a blocking antibody, an Erk inhibitor and/or an EGR1 inhibitor.

In accordance with one embodiment a method of treating an inflammatory disorder, such as a chronic respiratory disorder, in a subject is provided. In one embodiment the method comprises the step of administering to the subject in need thereof an endothelial monocyte activating polypeptide II (EMAP II) inhibitor. In one embodiment the chronic respiratory disorder is bronchopulmonary dysplasia. In one embodiment cell specific inflammatory and proliferative consequences of bioactive sphingolipid S1P signaling pathway are reduced by administering an EMAP II inhibitor, wherein the inhibitor is selected from an EMAP II signaling pathway inhibitor, a blocking antibody, an Erk inhibitor and/or an EGR1 inhibitor.

In accordance with one embodiment a method of treating a smooth muscle proliferation disorder in a subject is provided. In one embodiment the method comprising the step of administering to the subject in need thereof an endothelial monocyte activating polypeptide II (EMAP II) inhibitor. In one embodiment the smooth muscle cell proliferation disorder is a vascular disorder, including but not limited to atherosclerosis, restenosis, or pulmonary hypertension. In one embodiment cell specific inflammatory and proliferative consequences of bioactive sphingolipid S1P signaling pathway are reduced by administering an EMAP II inhibitor, wherein the inhibitor is selected from an EMAP II signaling pathway inhibitor, a blocking antibody, an Erk inhibitor and/or an EGR1 inhibitor.

In one embodiment the EMAP II inhibitor comprises an antibody that specifically binds to EMAP II. Antibodies that specifically bind to EMAP II are known to those skilled in the art and include monoclonal antibodies and fragments thereof. In one embodiment monoclonal antibodies can be injected intravenously or parentally using standard techniques known to the skilled practitioner. In accordance with one embodiment the activity of EMAP II can be inhibited by the subcutaneous administration of EMAP II mAb. The administration of monoclonal antibodies via parenteral route has been demonstrated to effectively reduce in vivo activity of EMAP II as reported in Skalko-Basnet et al, Biologics: “The role of delivery systems in improved therapy”, Biologics. 2014; 8: pp 107-114: and Hongyan Lu et al, Molecular Therapy August 2018, Vol. 26 No 8, p1867-2076, the disclosures of which are expressly incorporated herein.

In one embodiment the subcutaneous route of administration is used as this route gives a patient an easy opportunity for self-administration as opposed to intravenous administration in an acute care setting.

In one embodiment the level of EMAP II activity is reduced in a patient by administering an siRNA that targets the EMAP II transcript. The nucleic acid sequence of EMAP II is known and those skilled in the art have the required skills to produce an siRNA effective in reducing overall EMAP II activity in vivo or in vitro.

In accordance with one embodiment the level of EMAP II activity is reduced in a patient by interfering with the cleavage of Aminoacyl tRNA synthetase complex Interacting Multifunctional Protein 1 and thus reducing the levels of EMAP II present in a patient. In one embodiment the method comprises administering a caspase protein based inhibitor, including for example the commercially available inhibitor Z-ASTD-FMK: having the structure:

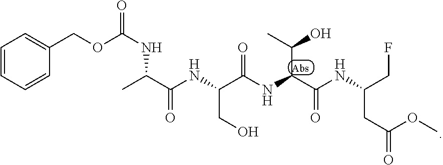 [SMILES: COC(=O)C[C@@H](CF)NC(=O)[C@@H](NC(=O)[C@H](CO)NC(=O)[C@H](C)NC(=O)OCc1ccccc1)[C@@H](C)O]

### Example 1

Materials.

The primary antibodies STAT3, pSTAT3 (Y705), ERK1/2, pERK1/2, SphK1, EGR1, Lamin B1 and cyclin 1 were from Cell Signaling Technology (CST) (Danvers, Mass.), SphK1 and alpha-tubulin was from Abcam and phospho-SphK1 (Ser225) was purchased from ECM Biosciences. HRP-conjugated rabbit and mouse secondary antibodies were from CST, Alexa fluorescent labeled secondary antibodies and phalloidin were purchased from Thermo Fischer Scientific (Waltham, Mass.). Inhibitors W 146 (iS1PR1), JTE 013 (iS1PR2), TY 52156 (iS1PR3) and PF543 (iSphK1) were from Cayman Chemical (Ann Arbor, Mich.), PD 184161 (iERK) was from Santa Cruz Biotechnology (Dallas, Tex.) and Ruxolitinib (iJAK1//2) and Trametinib (iERK1) were purchased from LC labs (Woburn, Mass.). EMAP II neutralizing antibody prepared by applicant (Anti-EMAP II) was used to chemically block EMAP II activity.

Emap II Preparation.

6x-His tagged EMAP II was prepared as previously described (Schwarz et al., 2000). The endotoxin levels in all preparations used for this study were below detectable limits, containing<0.1 ng/EU (GenScript).

Cell Culture and Treatments.

RAW 264.7 (ATCC) mouse macrophages and Human Embryonic Kidney (HEK) 293T cells were cultured in DMEM media with 10% fetal bovine serum (PBS) and glutamine HEPES buffer and beta-mercaptoethanol were added to the growth medium used for RAW 264.7 macrophages. Primary human pulmonary artery smooth muscle cells (hPASMC) purchased from Lonza (Walkersville, Md.) were cultured in complete growth medium containing smooth muscle growth media-2 (SmGM-2) with 10% FBS and growth factors provided as a kit by the supplier (Lonza, (Walkersville, Md.). THP-1 human monocyte cells were cultured in RPMI 1640 with 10% FBS and glutamine Cells were cultured in a humidified atmosphere with 5% CO2 at 370 C. For all studies passages 5-14 were used for RAW 264.7 and passages 5-10 were used for hPASMCs. For treatment studies, subconfluent cells plated in multi-well plates were serum starved and if required, pretreated for with inhibitors before stimulating with 2 μg/mL recombinant EMAP II.

Immunoblotting.

After appropriate treatments, protein lysates were prepared by using RIPA buffer supplemented with phosphatase and protease inhibitors (Thermo Fischer Scientific). Alpha-tubulin was used as a loading control. For nuclear and cytoplasmic fractionation, NE-PER kit from Thermo Fischer Scientific was used. Western blots were performed according to standard methods and quantified using densitometry using Image Studio Lite software from LI-COR Biosciences.

Transfection and luciferase assay. Human Embryonic Kidney (HEK) 293-T cells cultured in DMEM media containing 10% serum were reverse co-transfected with pGL4.47fluc2P/SIE/Hygrol and pGL4.74 [hRluc/TK] using Attractene Transfection Reagent (301005, Qiagen) as per manufacturer's protocol. Pretreated with inhibitors for 1 hour as required. After the treatment cells were incubated for 24 hours and luciferase activity was measured using Dual-Glo Luciferase Assay (E2920, Promega) per manufacturer's guidelines.

Differentiation of THP-1 Cells.

Few passages after thawing of THP-1 cell line, cells were seeded in Nunc Lab-Tek™ II 4-well imaging plates and treated with 100 nM Phorbol myristate acetate (InvivoGen, CA) for 3 days allowing THP-1 monocytes to differentiate into macrophages.

Immunofluorescence Staining

Primary hPASMCs cells or human THP-1 macrophages were seeded in Nunc Lab-Tek™ II 4-well imaging plates (Thermo Fisher Scientific) chamber well slides and cultured in growth medium. After the serum starvation cells were stimulated with EMAP II. Treated cells were rinsed with PBS and fixed with 4% paraformaldehyde at room temperature for 15 min and permeabilized with 0.1% Triton X-100 at room temperature for 10 min After washing with PBS three times, the cells were incubated with EGR1, 1amin, or pSphK1 antibody at 40C overnight. The cells were then rinsed with PBS three times and subsequently incubated with respective secondary antibody conjugated with Alex Fluor 647 and 488 or Phalloidin 488 at room temperature for 1 h. The cells were rinsed with PBS three times and coverslips were mounted with SlowFade Gold Antifade Mountant with DAPI (Thermo Fischer Scientific) and the cells were examined under Olympus microscope with 40× for EGR1 expression and with 60× for pSphK1 water immersion.

Transfection with Small Interfering RNA.

Knockdown of endogenous EGR1 was carried out by transfecting with 25 nM final concentration of ON-TARGETplus siRNAs specific for EGR1 or non-targeting control purchased from GE Dharmacon (Lafayette,CO) using Viromer Blue (Lipocalyx) per manufacturer's instructions. Cells were stimulated with EMAP II 48 h post-transfection.

TABLE 2
siRNA constructs

| Gene | Sequence | Vendor | Catalog No. |
|---|---|---|---|
| Human EGR1 | GAUGAACGCAAGAGGCAUA | Dharmacon | L-006526-00-0005 |
| | (SEQ ID NO: 3) | | |
| | CGACAGCAGUCCCAUUUAC | | |
| | (SEQ ID NO: 4) | | |
| | GGACAUGACAGCAACCUUU | | |
| | (SEQ ID NO: 5) | | |
| | GACCUGAAGGCCCUCAAUA | | |
| | (SEQ ID NO: 6) | | |
| Non-targeting Control | UGGUUUACAUGUCGACUAA | Dharmacon | D-001810-10-05 |
| | (SEQ ID NO: 7) | | |
| | UGGUUUACAUGUUGUGUGA | | |
| | (SEQ ID NO: 8) | | |
| | UGGUUUACAUGUUUUCUGA | | |
| | (SEQ ID NO: 9) | | |
| | UGGUUUACAUGUUUUCCUA | | |
| | (SEQ ID NO: 10) | | |

Analysis of SIP, Sphingosine and Ceramides by LC-MS/MS.

Collected cells were rinsed with PBS, pelleted and frozen at minus 80° Celsius. Sphingolipids extraction and analysis is carried out at the Medical University of South Carolina (MUSC) core facility using eight-point calibration curves generated for each target analyte by High Performance Liquid Chromatography-Tandem Mass Spectrometry (HPLC/MS/MS) system operating in positive multiple reaction-monitoring (MRM) mode engaging a gradient elution. Sphingolipids are normalized to inorganic phosphate levels determined from the Bligh & Dyer lipid extraction method. Biological samples were from different passage numbers and on different days.

Cell Proliferation Assay.

Cell proliferation of hPASMCs was assessed using WST-1 cell proliferation reagent (Roche Diagnostic Corporation, IN) in a 96-well plate. Human PASMCs were seeded at 4200 cells/well density in 100 μl complete medium and serum starved for overnight. Cells were stimulated for 48 hours with 2 μg/mL EMAP II following the pretreatment with inhibitors. WST-1 reagent (10 μL) was added into each well and incubated for 90 minutes in a humidified atmosphere with 5% CO2 at 370 C. Absorbance was measured at 450 nm using microplate reader. Blank control well was with medium and WST-1 reagent without cultured cells. All samples were in duplicates. Cell proliferation was determined by subtracting the background reading from average of duplicate of each sample.

RNA Extraction and Quantification.

Total RNA was extracted with TriZol using standard RNA extraction protocol and reverse transcribed with SuperScript III Reverse Transcriptase (Thermo Fischer Scientific). SphK1, Tnf, Il6, EGR1, Gapdh, Hprt1 transcripts were quantified using PrimePCR SYBR Green assays from BioRad.

Statistical Analysis.

The data are presented as means±S.E. from at least three independent experiments. Statistical significance was determined with unpaired Student's t-test using GraphPad Prism software.

### Results

EMAP II Induced STAT3 Activation in Macrophages is Linked with S1P Signaling and Erk Activation.

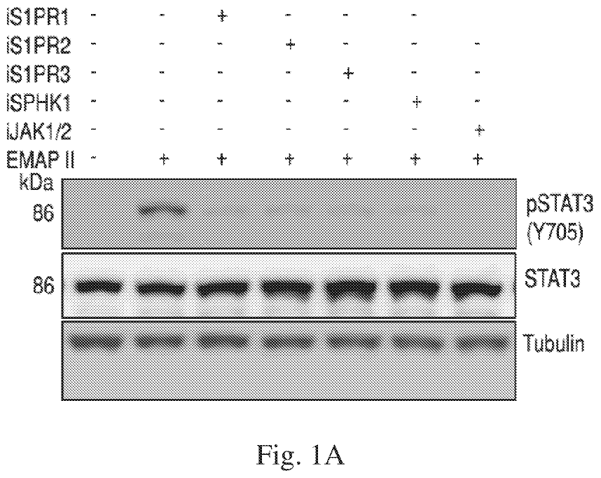

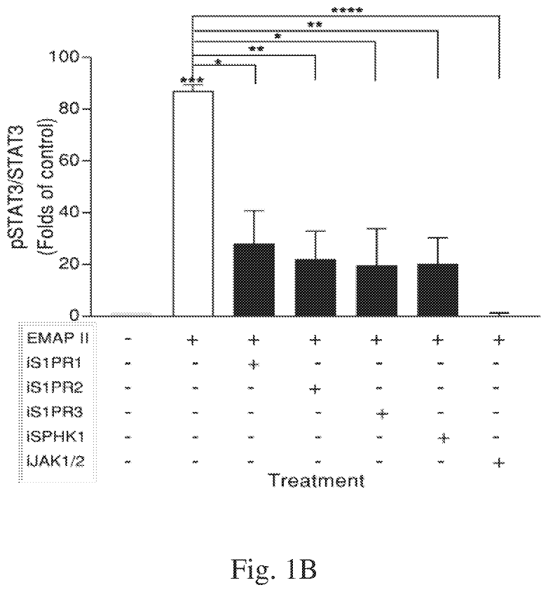

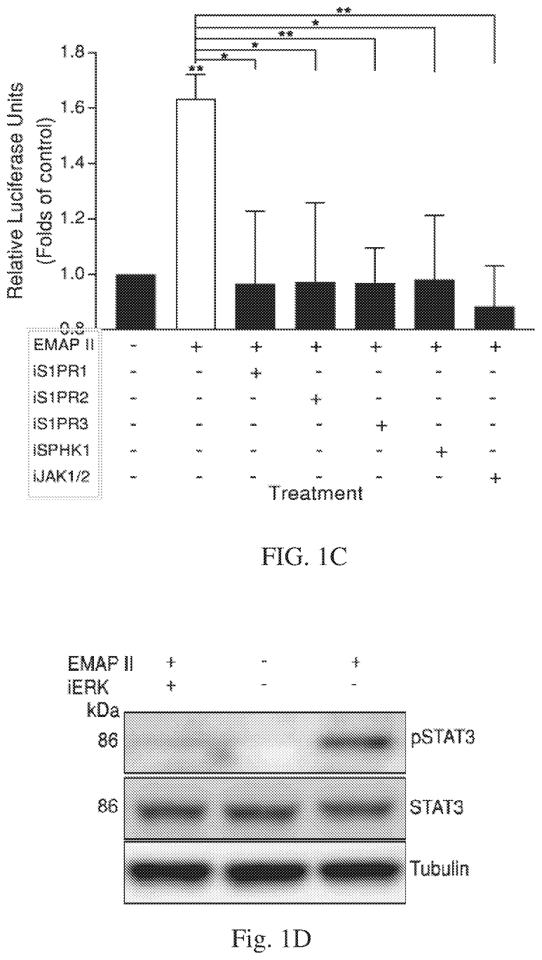

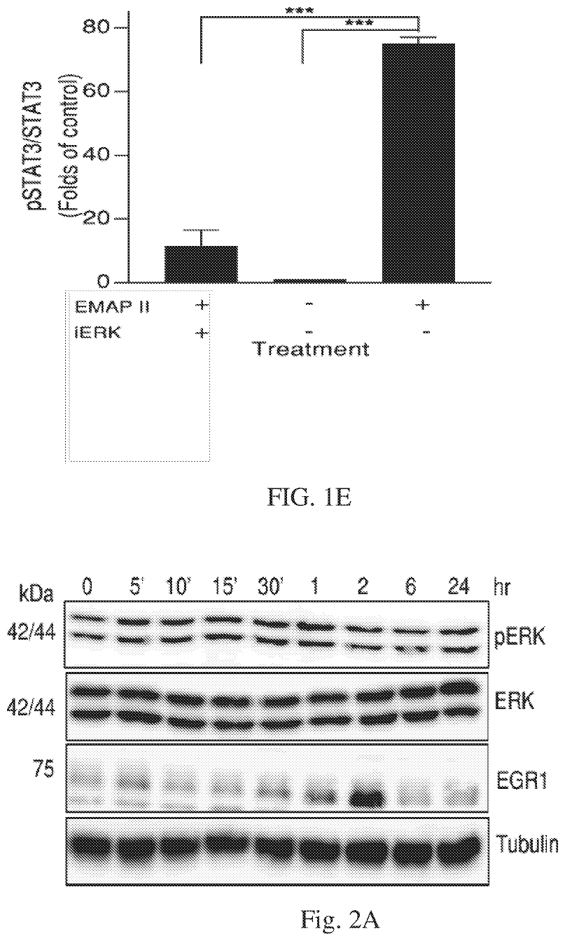

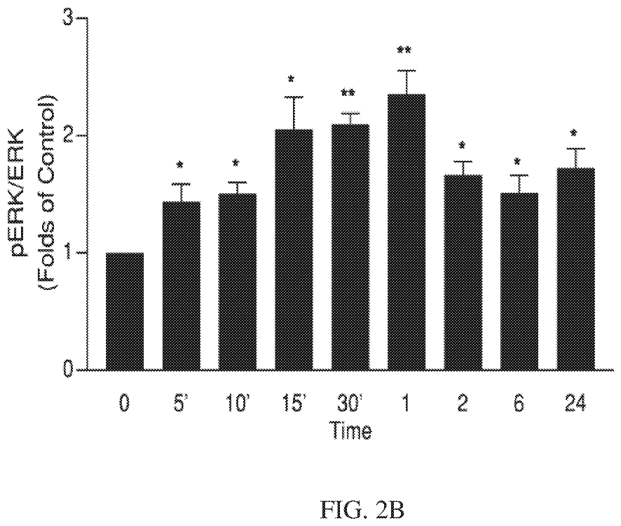

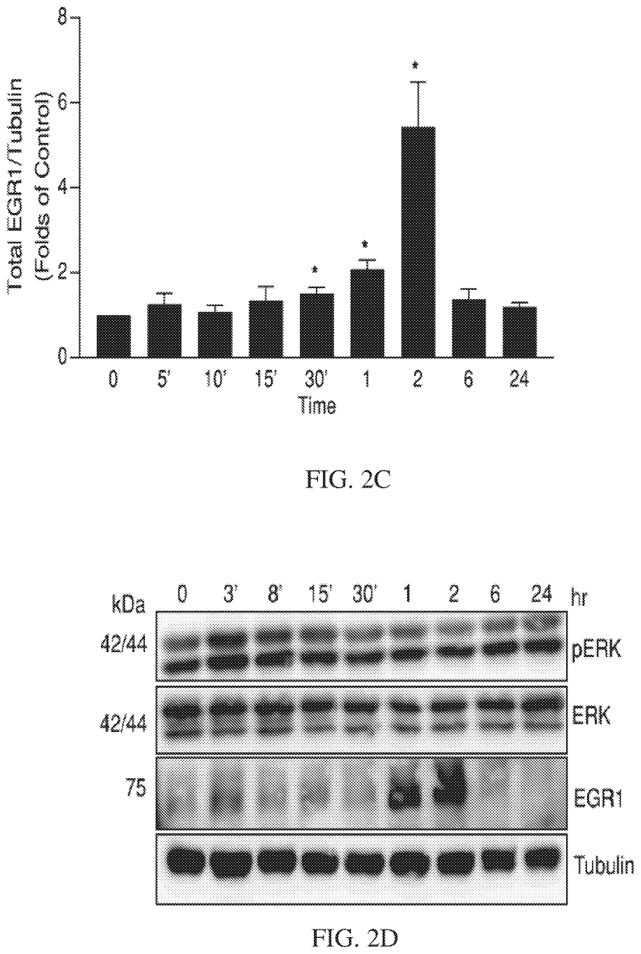

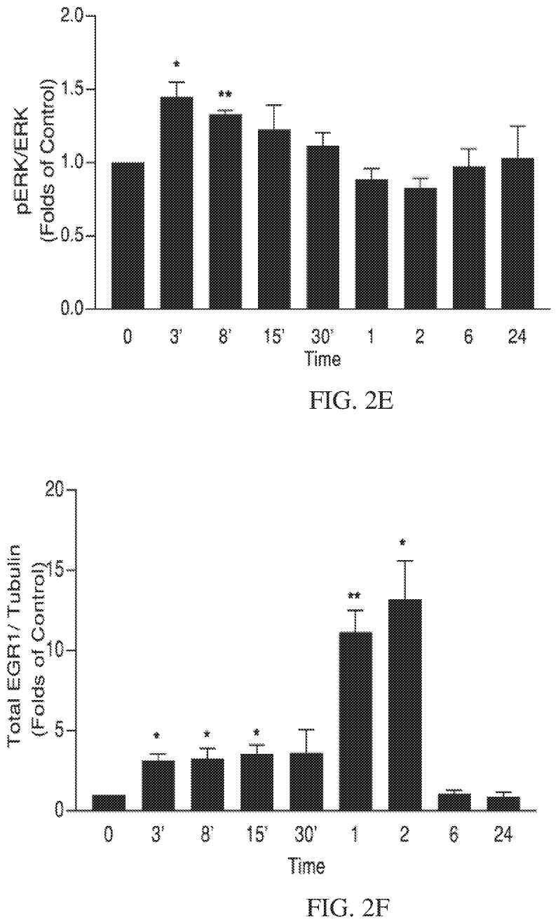

Previous studies demonstrated that EMAP II induces a JAK dependent biphasic tyrosine phosphorylation of STAT3 (Y705) and increases STAT3 mediated transcriptional target genes in macrophages. Interestingly, the EMAP II induced STAT3 mediated gene transcripts paralleled several of the known S1P mediated gene transcripts. The sequential steps and contribution of S1P signaling axis in EMAP II signaling was explored. EMAP II induced STAT3 activation in macrophages while pretreatment with S1PR specific inhibitors [iS1PR1 (S1PR1 inhibitor, 1 μM), iS1PR2 (S1PR2 inhibitor, 1 μM), iS1PR3 (S1PR3 inhibitor, 1 μM)], SphK1 inhibitor [iSphK1, 100 nM)] as well as a JAK1/2 inhibitor [iJAK1/2, 1 μM] significantly inhibited EMAP II induced pSTAT3 (FIGS. 1A and 1B). These findings were further supported as luminescence of STAT3 transcription reporter plasmid (FIG. 1C) demonstrated that EMAP II induced STAT3 activation is linked with the SIP signaling axis. Chemical blockade of SphK1 significantly inhibited EMAP II induced STAT3 activation and luminescence of STAT3 transcription reporter plasmid (FIGS. 1A, 1B and 1C) suggesting that EMAP II signaling was upstream of S1PR. As EMAP II is a promiscuous mediator of tyrosine kinase signaling, the impact of EMAP II on a common tyrosine kinase-signaling factor was tested, ERK1/2. EMAP II treatment induced ERK1/2 phosphorylation in macrophages (FIG. 2A) while pretreatment with a chemical inhibitor to block phosphorylation of ERK1/2 (iERK: MEK inhibitor, 10 μM) significantly inhibited EMAP II induced pSTAT3 (FIGS. 1D and 1E) suggesting that pSTAT3 activation occurs primarily through EMAP II initiated ERK1/2 mediated signaling cascade.

### EMAP II Induces Time Dependent Bi-Modal ERK Activation and Overexpression of Immediate Early Transcription Factor EGR1.

To determine the impact and timing of EMAP II induced ERK1/2 phosphorylation, a 24-hour time course examined the impact of EMAP II in macrophages and SMC on pERK1/2. A time dependent statistically significant bi-modal activation of pERK1/2 (normalized to ERK1/2) in macrophages was investigated(FIGS. 2A and 2B) as early as 5 minutes and reached the peak at 1 hour before subsiding only to again be noted to increase at 24 hours. This observation was not limited to macrophages alone as pulmonary SMCs also demonstrated similar time dependent bi-modal ERK1/2 activation in response to EMAP II (FIGS. 2D and 2E) where EMAP II induced pERK1/2 reached the peak as early as 3 minutes, subsided and started rising back again at 24 hours. Since EMAP II signaling is intertwined around both ERK activation (FIGS. 1D, 1E, 2A, 2B, 2D and 2E) and SphK1 (FIGS. 1A, 1B and 1C), the effect of EMAP II on an immediate early transcription factor, EGR1 known to interact with both ERK1/2 and SphK1 transcription was explored. Protein levels of EGR1 in whole cell lysates from the time course study in both macrophages and SMCs demonstrated a significant upregulation of EGR1 expression at 1 to 2 hours following EMAP II treatment in macrophages and SMCs (FIGS. 2A, 2C, 2D and 2F).

### EMAP II Induced Upregulation of Nuclear EGR1 Expression is Mediated Through ERK Activation.

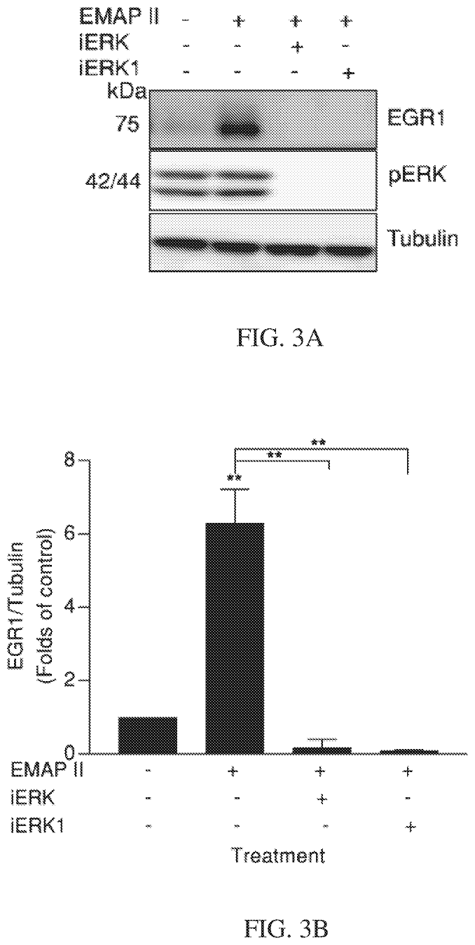

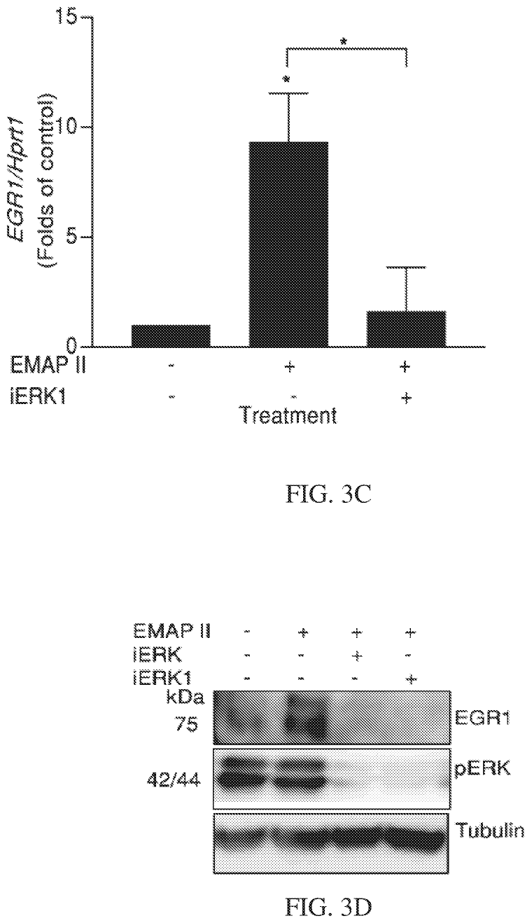

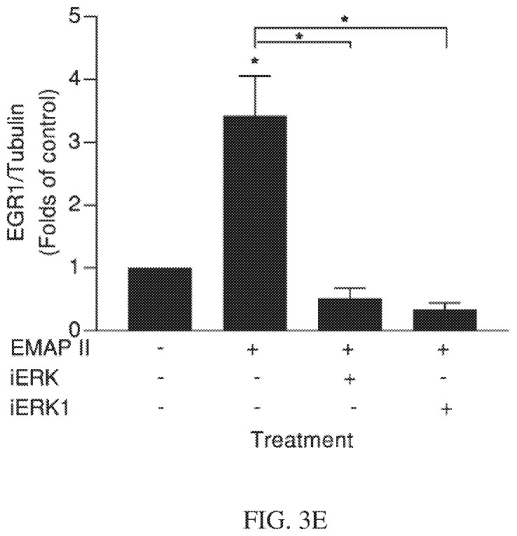

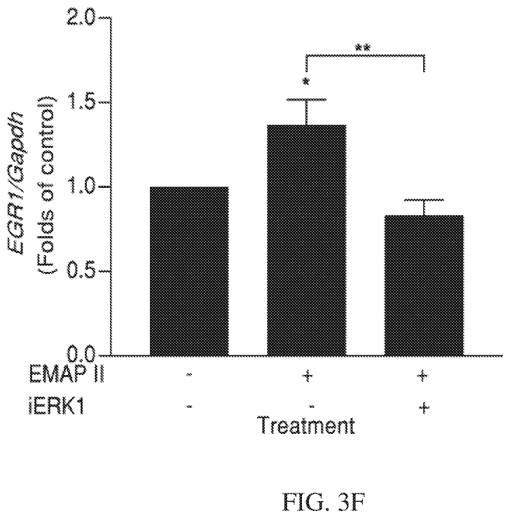

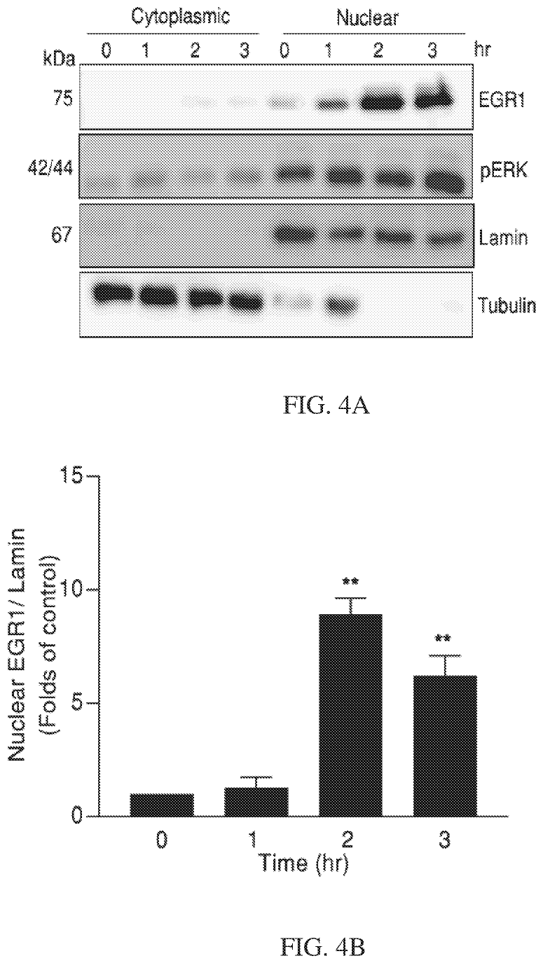

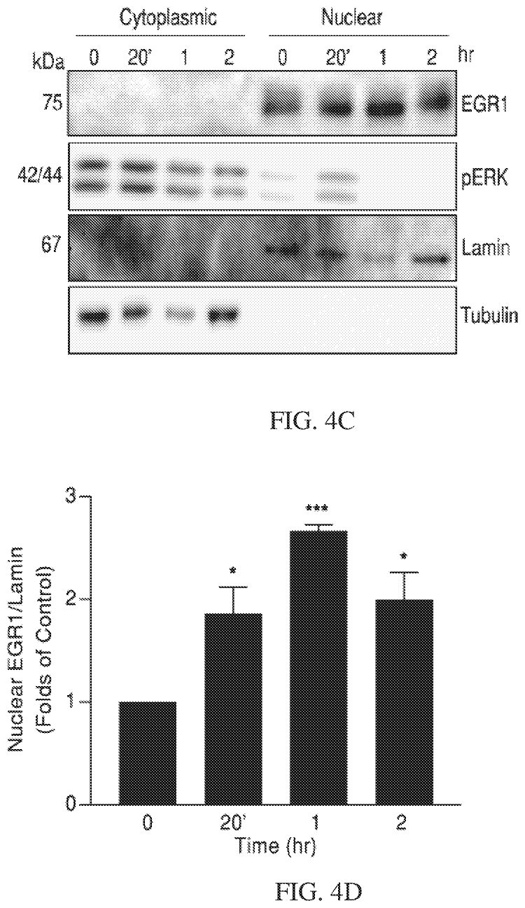

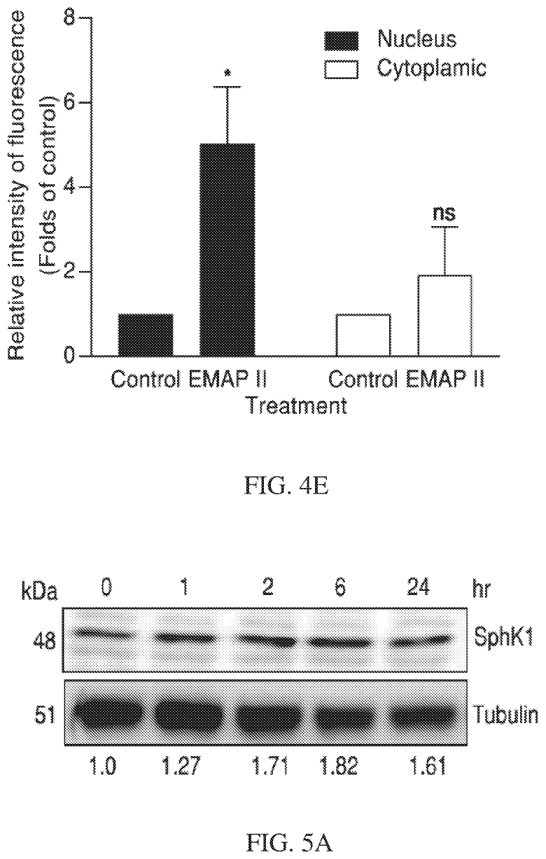

Previous studies have identified nuclear pERK1/2 as an important transcriptional regulator of EGR1. Utilizing nuclear and cytoplasmic fractions, the impact of EMAP II treatment on pERK1/2 and EGR1 nuclear expression in macrophage and SMC populations was studied. Consistent with an pERK1/2 mediated ERG1 transcription, MEK inhibition by iERK (MEK inhibitor, 10 μM) and iERK1 (MEK inhibitor, 10 μM) blocked EMAP II induced EGR1 in both macrophages (FIGS. 3A and 3B) and SMCs (FIGS. 3D and 3E). To determine whether EGR1 transcription is mediated through ERK activation, EGR1 transcripts were quantified in macrophages and SMCs with or without iERK1 (MEK inhibitor, 10 μM) pretreatment following EMAP II stimulation and normalized against Hprt1 in macrophages (FIG. 3C) and Gapdh in SMCs (FIG. 3F). Results demonstrated EMAP II induced EGR1 transcription can be inhibited by blocking ERK1/2 phosphorylation. Furthermore, EMAP II significantly (9-fold) upregulated nuclear expression of EGR1 in macrophages at 2 hours (FIGS. 4A and 4B) and in SMCs EMAP II upregulated EGR1 expression (2.5-fold) at 1 hour before subsiding (FIGS. 3C and 3D) Immunofluorescent analysis of SMCs stimulated with EMAP II for 1 hour demonstrated significantly elevated nuclear EGR1 expression as indicated by co-localization of EGR1, DNA and 1amin as compared to control (FIG. 4E). These results suggest that EMAP II upregulated EGR1 may play a major role as a transcription factor in EMAP II mediated signaling cascade.

### EMAP II Upregulates SphK1 Transcription and Translation Through EGR1.

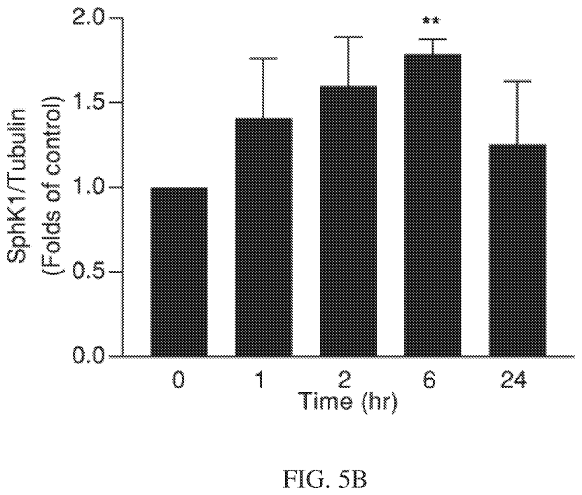

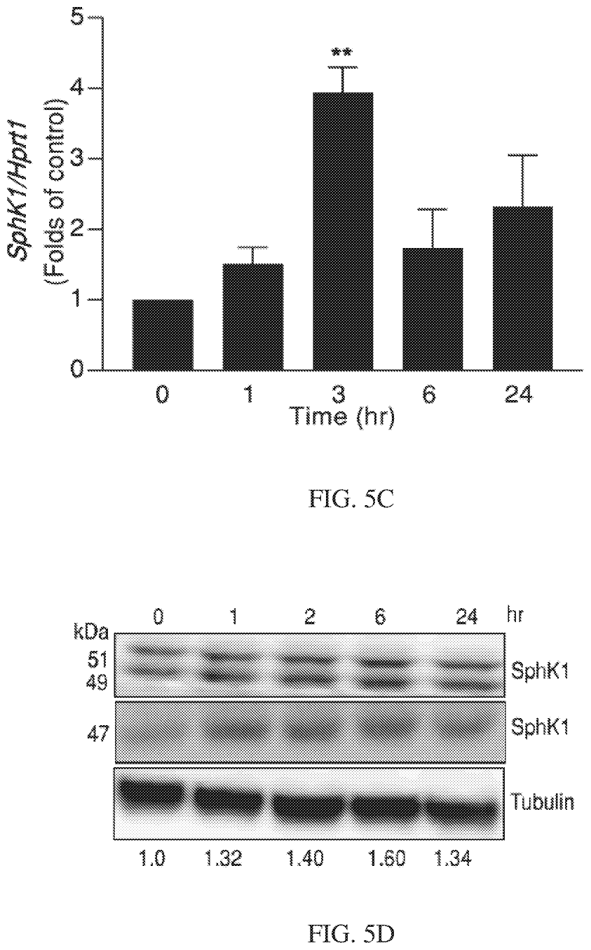

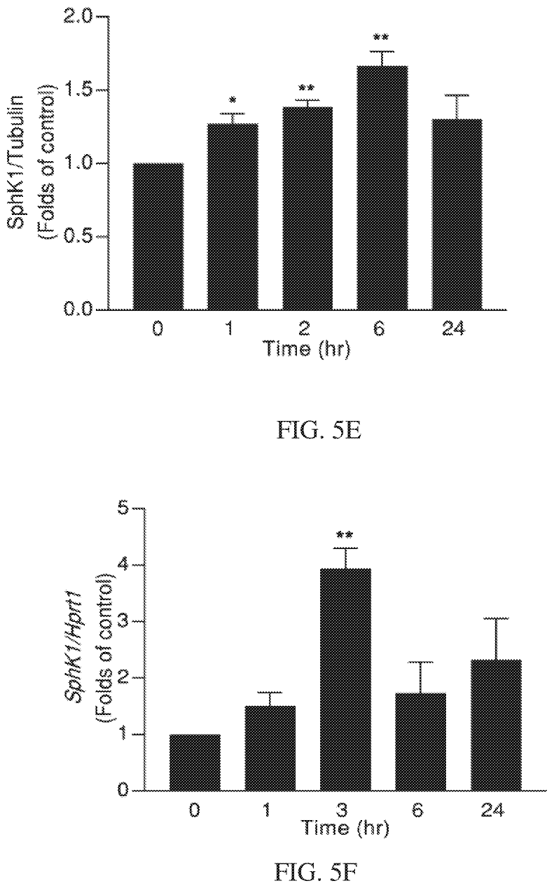

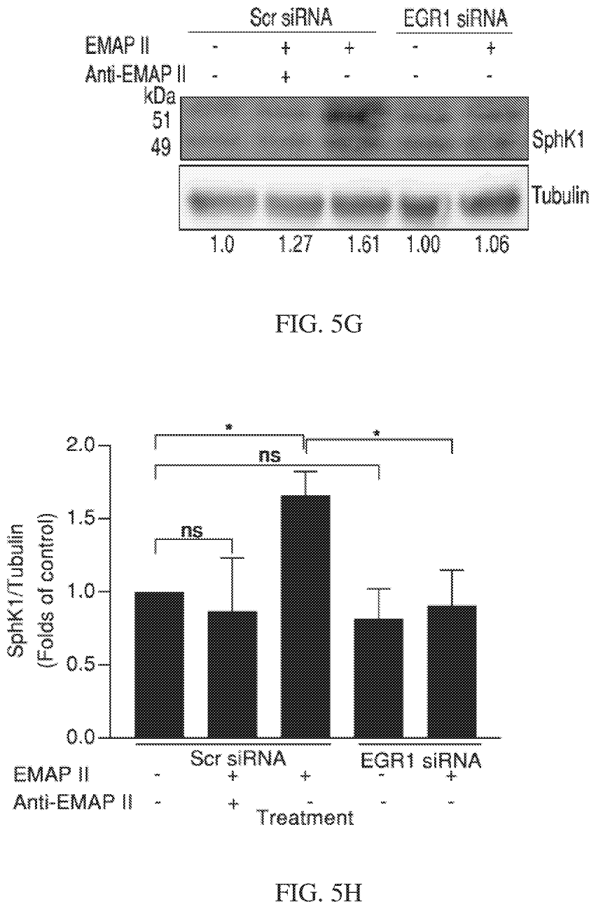

As EGR1 has been previously identified as a SphK1 transcriptional mediator, the effect of EMAP II signaling on SphK1 protein levels were explored. EMAP II induces a significant overexpression of SphK1 at 6 hours in both macrophages (FIGS. 5A and 5B) and SMCs (FIGS. 5D and 5E). To determine the effect of EMAP II on SphK1 transcription, q RT-PCR of SphK1 transcription was performed. EMAP II induced a time-dependent increase in SphK1 transcription that peaked at 3 hours after EMAP II stimulation in both macrophages and SMC types (FIGS. 5C and 5F). Furthermore, knockdown of EGR1 using specific siRNA and blocking EMAP II activity by pretreating with EMAP II neutralizing antibody significantly attenuated EMAP II induced SphK1 protein upregulation at 6 hours (FIGS. 5G and 5H) and upregulation of transcript level at 3 hours (FIG. 5I).

Successful knockdown of EGR1 was confirmed by immunoblotting (FIGS. 5J and 5K). EMAP II induces bi-modal phosphorylation of SphK1. Having established that SphK1 is upregulated by EMAP II through EGR1, it was sought to determine whether EMAP II affects the activation of SphK1. Since phosphorylation of SphK1 at Ser225 is required for SphK1 activity, the pSphK1 protein expression after EMAP II stimulation in specific time points was initially studied Immunoblotting demonstrated that EMAP II induced bi-modal SphK1 phosphorylation at 1 and 6 hours in SMCs (FIGS. 6A and 6B). SphK1 resides in the cytoplasm where upon phosphorylation by ERK1/2, it translocate to the plasma membrane(Jarman et al., 2010). EMAP II stimulated translocation of pSphK1 to the plasma membrane in SMCs and THP1 macrophages.

Immunostaining for pSphK1 and actin suggests that after 15 minutes and 1 hour EMAP II stimulation pSphK1 is trafficked to the plasma membrane as compared to the control in SMCs. In THP1 macrophages elevated levels of pSPHK1 was observed near the cell membrane at 1 hour of EMAP II treatment as indicated by actin staining (plasma membrane). In addition, it was observed that in SMCs after 6 hours of EMAP II treatment, pSphK1 is trafficked to the membrane demonstrating bi-modal phosphorylation of SphK1 at 1 and 6 hours as suggested by the immunoblotting for pSphK1.

EMAP II Modulates S1P Levels.

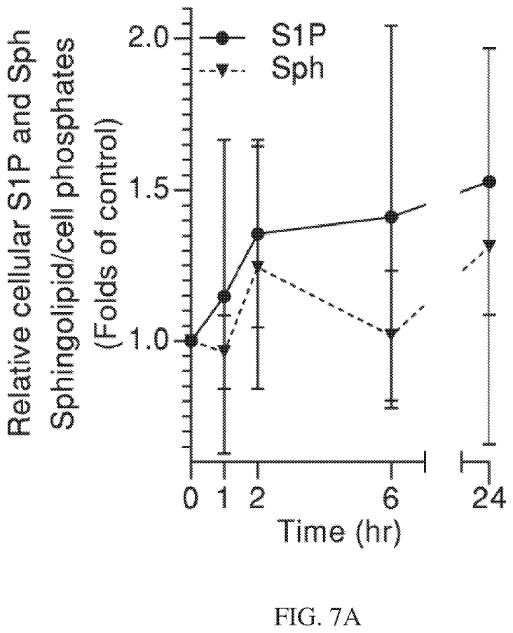

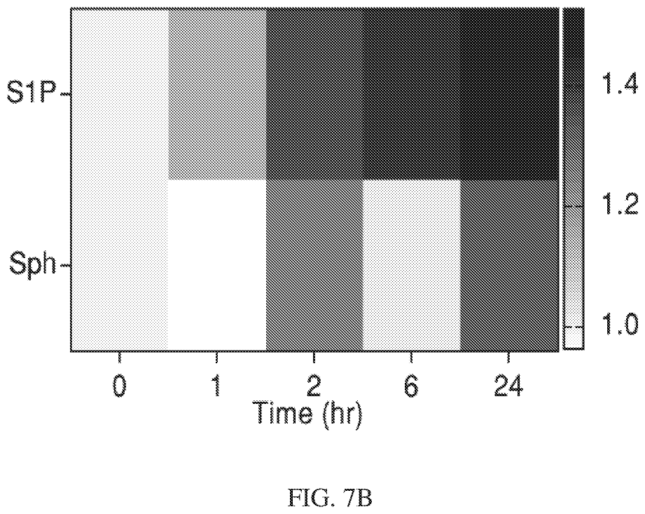

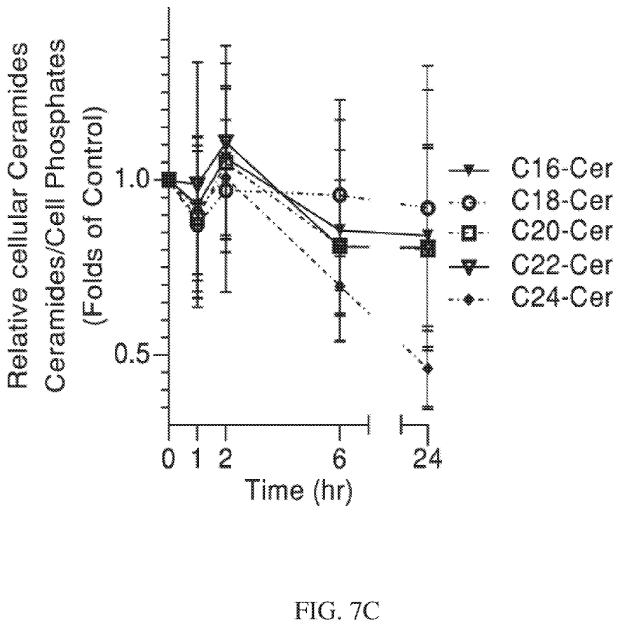

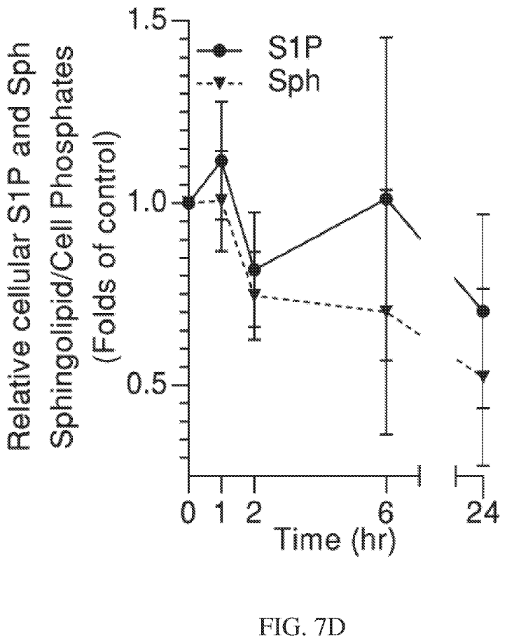

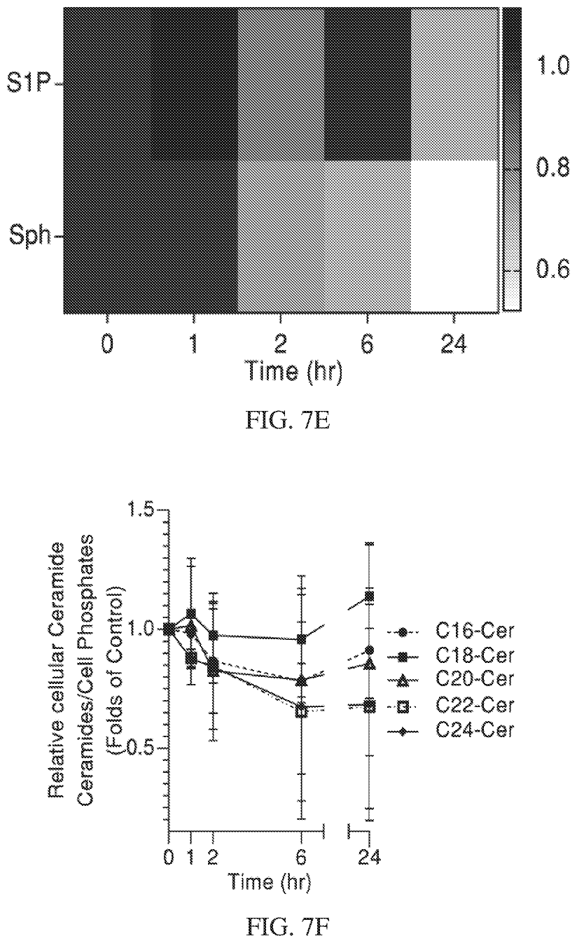

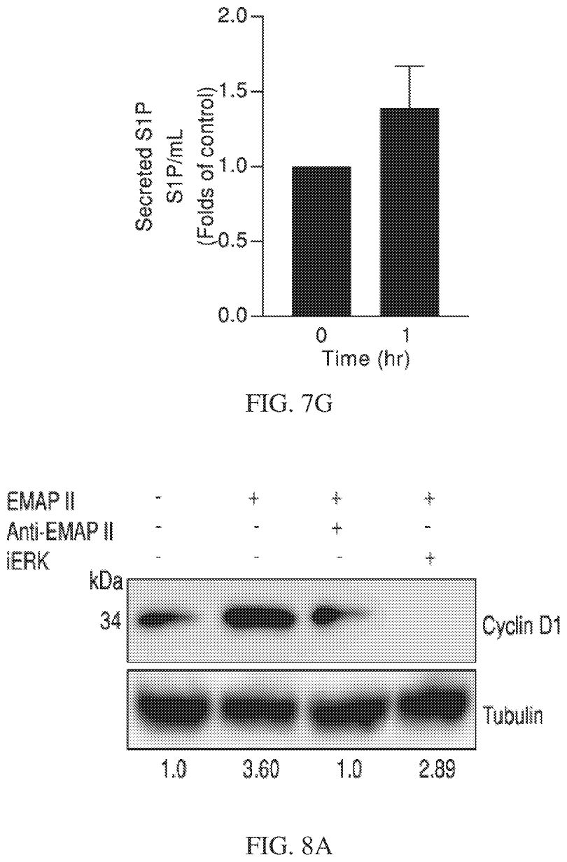

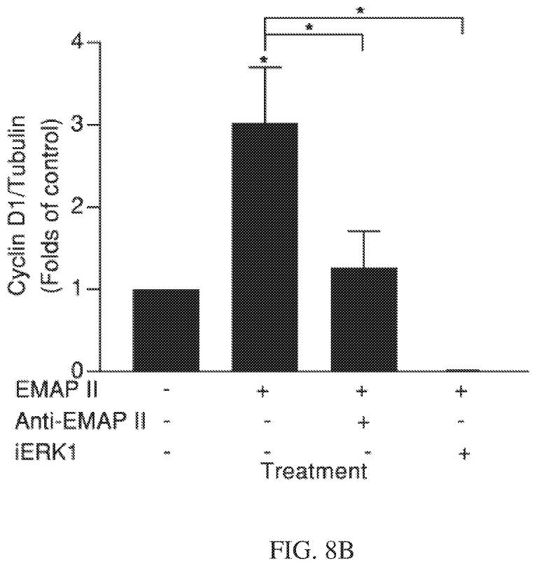

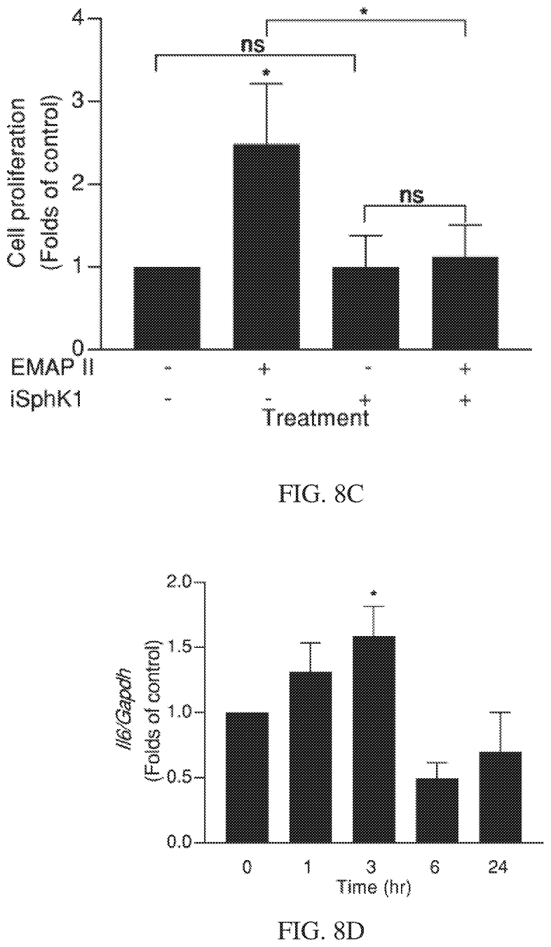

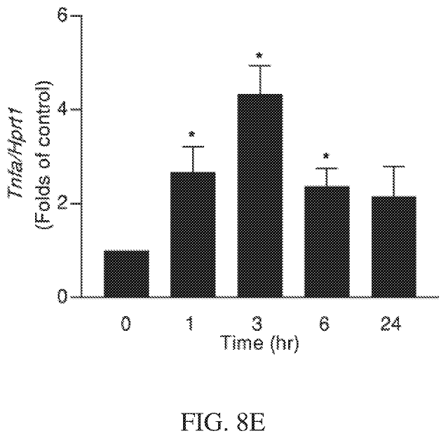

Corresponding with EMAP II induced trafficking of pSphK1 to the plasma membrane, measurements of cellular sphingosine, S1P, and ceramide levels by HPLC-MS/MS in macrophages and SMCs after EMAP II treatment (normalized against inorganic phosphates) showed a time dependent increase in S1P levels (FIGS. 7A, 7B, 7D and 7E) and a reduction in S1P precursors sphingosine (FIGS. 7A, 7B, 7D and 7E) and ceramides (FIGS. 7C and 7F). The S1P measurements in macrophages showed a continuous elevation throughout 0-24 hours time course while its precursors sphingosine and ceramides show a bi-modal depreciation (FIGS. 7A, 7B,7C). In SMCs, cellular S1P measurements showed a bi-modal elevation at 1 and 6 hours and cellular sphingosines and ceramides measurements showed the depreciation throughout the time (FIGS. 7D, 7E and 7F). Extracellular S1P in SMCs showed an elevation after 1 hour of EMAP II treatment compared to the control (FIG. 7G). The downstream effects of EMAP II/S1P signaling are cell type specific. Although EMAP II signaling activates STATS in macrophages as shown in FIG. 1, EMAP II does not impact pSTAT3 levels in SMCs (data not shown). Moreover, EMAP II induced tumor necrosis factor (TNF) cytokine production in macrophages as demonstrated by increased TNF transcription level (FIG. 8E) while this observation was absent in SMCs (data not shown). EMAP II modulates cyclin D1 in SMCs in an ERK1/2 dependent manner instead of TNF∞ after 48 hours of EMAP II stimulation (FIGS. 8A and 8B) which was confirmed by WST1 proliferation assay (FIG. 8C) suggesting that EMAP II promotes cell proliferation in SMCs. Importantly, inhibition of SphK1 ablated EMAP II promoted proliferation in SMCs (FIG. 8C) without affecting the basal cell proliferation rate. Likewise, increased transcription of interleukin 6 (Il6) which is a major proliferative marker in SMCs(Yao et al., 2007) was found in SMCs by q RT-PCR (FIG. 8D).

Recent studies have cemented that the altered sphingolipid S1P homeostasis is involved in several cardiovascular and pulmonary pathophysiological processes with two of the major characteristics being inflammation and arterial narrowing such as atherosclerosis, pulmonary hypertension, and lung disease of prematurity. A conserved and coherent upstream signaling link was observed between the protein EMAP II and lipid S1P in inflammatory cells and SMCs that initiates cell specific downstream pathophysiological functions that may contribute to the pathogenesis of alveolar simplification, pro-inflammation and pulmonary hypertension (PH). It was identified that 1) EMAP II induces bi-modal ERK1/2 activation and SphK1 activation, 2) EMAP II induced ERK1/2 results in overexpression of transcription regulator of differentiation, inflammation, and mitogenesis protein coding gene EGR1, 3) EMAP II induced elevated nuclear expression of EGR1 regulates SphK1 transcription, and 4) EMAP II promotes membrane localization of pSphK1 and increases S1P expression. Thus, identifying the upstream crosstalk between signals of SIP and EMAP II in downstream molecular and signaling consequences that stimulate macrophage pro-inflammatory mediators and SMC proliferation associated with development of cardiovascular and pulmonary disorders sheds new insight into the regulation of S1P homeostasis.

Atherosclerosis is a chronic inflammatory disease where excessive accumulation of lipids and immune cells form plaques which eventually results in narrowing down of arterial lumen. A clinical study identified S1P as a stronger and more robust biomarker of severe atherosclerosis due to its augmented levels of S1P in serum. However, studies show pro- and anti-atherosclerotic properties of S1P suggesting that S1P acts as a double-edged sword in pathology of atherosclerosis.

Moreover, S1P levels reported to be elevated in lung tissues of patients with pulmonary hypertension (PH) where increased transcription of SphK1 showed to play the critical role in disrupting the S1P homeostasis. SphK1 mediated increased proliferation of perivascular smooth muscle cells (SMCs) reported to cause PH. In addition, recent studies reported distinctly elevated levels of S1P in tracheal fluids during first days of postnatal life in preterm infants with bronchopulmonary dysplasia (BPD) which is a lung disease of prematurity and conversely, the knockdown or pharmacological inhibition of SphK1 reported to attenuate BPD in mouse models warranting that SphK1/S1P would be a potential signaling pathway to be targeted in BPD. Similarly, moonlighting roles for EMAP II in disease progression mediated by inflammation include mediating inflammation by immune cell trafficking, angiogenesis and apoptosis. Previous studies link EMAP II to endothelial cell apoptosis, a detrimental factor in atherosclerosis and increased expression of E- and P-selectins and Tnf-1 which results in monocyte and neutrophils attraction. Moreover, EMAP II mediated macrophage recruitment and pro-inflammatory cytokine production are seen in BPD mouse models. Contribution of mediators such as EMAP II to disease progression in conjunction with observations that there is a signaling inter-relationship between two members of this class implicates a protein mediated lipid regulatory mechanism, suggesting that the early observed (1 hr) pSphK1 may be the activator of secondary ERK1/2 activation in EMAP II/SphK1/S1P signaling pathway.

Observed was a similar bi-modal pattern in both ERK1/2 and SphK1 activation, suggesting an inter-dependent relationship between ERK1/2 and SphK1. ERK1/2 is one of the members of mitogen-activated protein kinase super family and is known to directly phosphorylate SphK1 at Ser225 residue. Additionally, SphK1 carries a docking site for ERK1/2. However, there is evidence showing that ERK1/2 activated SphK1 is capable of activating ERK1/2 back in a positive feedback mechanism. The phosphorylation of SphK1 at Ser225 by ERK1/2 activators such as phorbol esters and PDGF increases enzyme activity by 14-fold and mediates the translocation of the enzyme from the cytosol to the plasma membrane bringing it into proximity to its substrates. EMAP II phosphorylates SphK1 at Ser225 by acting as an activator of ERK1/2 which may eventually initiate the feedback loop between ERK1/2 and SphK1.

Interestingly, EMAP II induces overexpression of nuclear EGR1, another known target of ERK1/2 and transcription activator of SphK1. EGR1 is a 80-82-kD inducible zinc finger transcription factor that has also been known to regulate cell growth, proliferation and inflammation in lung. Identified as one of the early biomarkers of lung injury in preterm lambs, EGR1 mRNA levels were increased 120-fold within 30 minutes of injury. Moreover, pharmacological inhibition of EGR1 has been reported to attenuate pulmonary vascular resistance and right ventricular hypertrophy in animal models. Atherogenic factors have also been reported to induce EGR1 expression in the aorta resulting in vascular lesions in human and mouse. Experiments indicate that inhibition of EGR1 suppresses EMAP II mediated SphK1 transcription representing a potential mechanism of regulating downstream S1P generation. Since studies show that EMAP II signaling provokes a bi-modal early upstream SphK1 phosphorylation in addition to the later EGR1 mediated SphK1 transcription and activation, it is questionable whether inhibition of EGR1 alone would be a promising therapeutic target for impeding EMAP II/SphK1/S1P signaling in disease process associated with pulmonary hypertension such as BPD. However, ablating ERK1/2 phosphorylation has been reported to inhibit EGR1 induced pulmonary vascular proliferation in newborn PH calves models supporting the fact that the phosphorylation of ERK1/2 is a key regulator of both SphK1 phosphorylation and transcriptional regulation. Thus providing a critical target point in regulation of downstream sphingosine phosphorylation. In addition, about 4 times induction of SphK1 transcription in EMAP II treated macrophages was observed compared to the control. Nevertheless, the protein expression of SphK1 in macrophages was only about 1.7 times. One possibility for the difference between the fold change of transcripts and protein expression levels might be due to multiple factors that affecting the protein translation including the level of mRNA and its half-life, translational efficiency, and the stability of the interested protein.

Immunoblotting and immunofluorescence studies showed that EMAP II signaling directs SphK1 activation and navigates phosphorylated SphK1 towards the plasma membrane that was confirmed by bi-modal elevation of S1P levels and depreciation of its precursors sphingosine and Moreover, the depreciation of ceramides indirectly supports the observation of S1P elevation as they are interconvertible lipids and sphingolipid rheostat of S1P and ceramide is known to control the cell fate. The feedback inhibition from the early augmented S1P on the secondary S1P augmentation and transient transport of S1P out of the cells might be some reasons for the secondary S1P peak to be weak in SMCs. Studies indicate that one of the mediators of the translocation of activated SphK1 to the plasma membrane is calmodulin-related protein calcium and integrin-binding protein 1 (CIB1) which provides a plasma membrane docking site via myristoylation where it's retention facilitates sphingosine phosphorylation. High cytosolic Ca2+ detected upon the CIB1-SphK1 interaction is consistent with previous reports that showed EMAP II treatment causes redistribution of intracellular calcium stores into the cytosol. Therefore, future mechanistic studies to determine if CIB1 is the binding protein responsible for EMAP II induced pSphK1 translocation is warranted.

Although upstream signaling of EMAP II/ERK/EGR1/SphK1/S1P is conserved in both cell types of macrophages and SMCs, studies demonstrated cell type-specific downstream responses suggesting that downstream effects may depend on the cell type and the availability of different S1PRs. In macrophages, EMAP II signaling induces STATS activation via S1PRs which mediates inflammatory state as shown by prior studies while in SMCs, EMAP II signaling results in proliferative signaling by overexpressing cell cycle regulating proteins like cyclin D1 which is required for G1/S phase transition. EMAP II promotes proliferation in SMCs challenging the conventional known apoptotic function of EMAP II in endothelial cells and is consistent with previous reports of increased peri-vascular SMC EMAP II expression in infants with BPD. A recent study has shown that macrophage migration inhibitory factor upregulates cyclin D1 via ERK1/2 leading to SMC proliferation causing PH in animal models. Elevated signaling molecules recruit inflammatory cells such as neutrophils and macrophages. Macrophages are the prominent inflammatory cells in chronic inflammation due to its functions in promotion and resolution of inflammation, pathogen clearance, and tissue restoration following injury. Chronic inflammation mediated by macrophages can contribute to the arterial remodeling observed in the progression of disease process such as cardiovascular and pulmonary disorders. In atherosclerosis, SMC proliferation and differentiation have shown to be linked with the soluble factors secreted by macrophages. Similarly, the dynamic interplay between macrophages and SMCs in vascular diseases is well recognized. Therefore, the source of EMAP II that acts on SMCs to promote proliferation can be expressed and secreted in recruited macrophages suggesting a novel aspect of a complex signaling mechanism.

EMAP II as a Mediator of S1P Homeostasis.

However, the proteolytically cleaved protein EMAP II and the bioactive lipid SIP are signals that get triggered as early response chemokines prior to influx of inflammatory cells upon the cellular damage. Therefore, in physiological conditions although it is possible that S1P is activated prior to EMAP II, findings suggest that there is an intimate crosstalk between two biological molecules may initiate a feedback loop mechanism elevating cellular S1P concentration.

In summary, findings indicate that EMAP II can be a potential key mediator of the bioactive lipid SIP. EMAP II modulated S1P signaling promotes activation of key mechanistic signaling pathways that contribute to the pro-inflammatory and arterial remodeling characteristics found in cardiovascular and pulmonary disorders through a two-phase process. An upstream conserved protein initiated biphasic activation of the bioactive lipid S1P and a lipid mediated downstream cell specific signaling pathway that contributes to disease progression. EMAP II inhibitory molecules can be used in accordance with these studies as therapeutic entities useful for treating alveolar simplification, inflammation and arterial remodeling associated with inflammatory and vascular remodeling disorders.

### Example 2

### The Impact of SphK Bi-Modal Phosphorylation on Macrophage Inflammation and SMC Proliferation.

Regulating phosphorylation of SphK is key to mediating the downstream impact of phosphorylation of sphingosine to S1P that is released either intracellular or transported into the extracellular space via transporter Spns2. Importantly, phosphorylation of SphK1 occurs in two waves. Initially, existing cytoplasmic SphK1 is phosphorylated initiating S1P release followed by a delayed second phosphorylation following transcriptional/translational of newly synthesized SphK1. Of note, phosphorylation and transcription of SphK1 are initiated by the same phosphorylated kinase, ERK1/2. Although an early and delayed signaling system could provide separate downstream functions of the released S1P, little is known regarding the consequences of bi-modal pSphK1 initiated release of S1P and its signaling impact. What is known is that S1P binding to its S1PR can promote S1PR internalization followed by S1PR recycling back to the cell surface within several hours providing a feedback system that takes advantage of the S1P gradient. However the impact of staggered S1P release is poorly understood. While it is known that repetitive S1P signaling in macrophages is part of an autocrine/paracrine feedback loop involved in the persistent activation of STATS mediated transcription of pro-inflammatory mediators through the NF-TB-IL-6-STAT32 amplification cascade in cancer, less is known regarding the role prolonged S1P release has on macrophage and SMC signaling in BPD. Our data indicates that EMAP II induces an early and late SphK1 phosphorylation, induces pERK1/2 and EGR1 expression and mediates SphK1 transcription. Based on these findings, ERK1/2 blockade inhibited EMAP II induced ERK1 phosphorylation and ERG1 translation. Utilizing ERK1/2 inhibitors versus EGR1 inhibition, our studies are targeted to dissect out EMAP II induced bi-modal activation of S1P on pro-inflammatory signaling in macrophages and proliferation in SMC.

### Gender Differences:

Recent studies determined that there are gender specific differences in prognosis following premature birth and associated chronic lung disease. Our studies include the examination of gender as one aspect of analysis.

### Confirmation of SphK1 Ser225 Phosphorylation:

EMAP II mediated release of S1P via SphK1 is supported by our data that EMAP II mediated pERK1/2 is responsible for pSphK1 Ser225 and subsequent phosphorylation of sphingosine. These findings are also in line with recent studies that determined that Ser225 phosphorylation of SphK1 is associated with pulmonary hypertension. Using Western blot analysis and an SphK2 selective inhibitor ABC294640, we will determine if the other SphK enzyme, SphK2 is activated by EMAP II via phosphorylation at SphK2 p578. To confirm that EMAP II targets phosphorylation of sphingosine through phosphorylation of SphK1, some studies will utilize TEPM macrophages isolated from SphK1 KO mice (male and female, from Jackson Laboratories). Isolated macrophages exposed to EMAP II will be studied for phosphorylation of ERK1/2 and SphK1, SphK2, S1P levels (intracellular and secreted), and transcription/translation of SphK2, IL10, IL6, TNF, and IL1 as well as secretion of mentioned cytokines will be examined.

The impact of upstream ERK inhibition on EMAP II induced pro-inflammatory mediators in macrophages and proliferation in SMC will be investigated and a determination made as to if EMAP II bi-modal phosphorylation of SphK1 is ERK mediated. STAT3 modulated inflammatory mediators are transcribed and secreted in macrophages treated with EMAP II. Our data suggests that EMAP II through a conserved upstream ERK mechanism is responsible for activation of SIP and subsequent STAT3 transcription and translation of inflammatory mediators in macrophages while Cyclin D1 expression is increased in SMC. Using ERK inhibitors (Tramatenib and PD184161) and knockdown of ERK using siRNA we will determine if EMAP II induction of pro-inflammatory mediators is ERK1/2 γSphK1γ SIP mediated. Using partially activated TEPM macrophages isolated from female compared to male C57BL6J mice and RAW macrophages, phosphorylation of ERK1/2 and SphK1, S1P levels, and transcription/translation of SphK1, IL10, IL6, TNF, IL1 (ELISA of secreted cytokines will be measured) will be explored. Human pulmonary artery smooth muscle cells (hPASMC) will be utilized to determine the impact of ERK1/2 inhibition (via inhibitors and siRNA) impacts phosphorylation of ERK1/2 and SphK1, S1P levels (intracellular and secreted), Cyclin D1 transcription/translation expression, and proliferation using WST1 assay.

The impact of inhibiting the second part of the EMAP II initiated bi-modal phosphorylation of SphK1 through EMAP II mediated SphK1 transcription will be investigated. Previous studies suggest that ERK1/2 activation of ERG1 transcription/translation contributes to transcriptional/translational regulation of SphK1. Similarly, our data supports an EMAP II mediated ERK1/2 ERG1 SphK1 transcription/translation regulation of SphK1. Initial studies suggests that EMAP II is a mediator of pSphK and EGR1 induction as pre-treatment with an EMAP II neutralizing antibody reduced SphK1 and pSphK1 expression while EGR1 siRNA in hPASMC reduced EMAP II induction of Egr1 expression. Using this EGR1 siRNA strategy, knockdown of ERG1 allows us to inhibit the second half of EMAP II induced bi-modal pSphK1. A second strategy will use Actinomycin D (ActD) to inhibit transcription in cells treated with EMAP II. In hPASMC phosphorylation of ERK1/2 and SphK1, S1P levels, Cyclin D1 transcription/translation expression, and proliferation will be examined while in TEPM ERK1/2 and SphK1, S1P levels, and transcription/translation of SphK1, IL10, IL6, TNF, IL1 (ELISA of secreted cytokines will be measured) will be explored. A third approach will utilize TEPM macrophages isolated from ERG1 KO male and female mice (Jackson laboratories) and treated with EMAP II. In this macrophage study phosphorylation of ERK1/2 and SphK1, SIP levels (intracellular and secreted), and transcription/translation of SphK1, IL10, IL6, TNF, IL1 (ELISA of secreted cytokines will be measured) will be examined Identify the impact of upstream ERK inhibition on EMAP II induced pro-inflammatory mediators in macrophages and proliferation in SMC and determine if EMAP II bi-modal phosphorylation of SphK1 is ERK mediated. STAT3 modulated inflammatory mediators are transcribed and secreted in macrophages treated with EMAP II (FIGS. 11A-E and 4A-4C). Our preliminary data suggests that EMAP II through a conserved upstream ERK mechanism is responsible for activation of SIP and subsequent STAT3 transcription and translation of inflammatory mediators in macrophages while Cyclin D1 expression is increased in SMC. Using ERK inhibitors (Tramatenib and PD184161) and knockdown of ERK using siRNA we will determine if EMAP II induction of pro-inflammatory mediators is ERK1/2 SphK1 SIP mediated. Using partially activated TEPM macrophages isolated from female compared to male C57BL6J mice and RAW macrophages, phosphorylation of ERK1/2 and SphK1, S1P levels, and transcription/translation of SphK1, IL10, IL6, TNF, IL1 (ELISA of secreted cytokines will be measured) will be explored. Human pulmonary artery smooth muscle cells (hPASMC) will be utilized to determine the impact of ERK1/2 inhibition (via inhibitors and siRNA) impacts phosphorylation of ERK1/2 and SphK1, S1P levels (intracellular and secreted), Cyclin D1 transcription/translation expression, and proliferation using WST1 assay. Impact of inhibiting the second part of the EMAP II initiated bi-modal phosphorylation of SphK1 through EMAP II mediated SphK1 transcription. Previous studies suggest that ERK1/2 activation of ERG1 transcription/translation contributes to transcriptional/translational regulation of SphK1. Similarly, our preliminary data supports an EMAP II mediated ERK1/2 ERG1 SphK1 transcription/translation regulation of SphK1. Initial studies suggests that EMAP II is a mediator of pSphK and EGR1 induction as pre-treatment with an EMAP II neutralizing antibody reduced SphK1 and pSphK1 expression while EGR1 siRNA in hPASMC reduced EMAP II induction of Egr1 expression. Using this EGR1 siRNA strategy, knockdown of ERG1 allows us to inhibit the second half of EMAP II induced bi-modal pSphK1. A second strategy will use Actinomycin D (ActD) to inhibit transcription in cells treated with EMAP II. In hPASMC phosphorylation of ERK1/2 and SphK1, S1P levels, Cyclin D1 transcription/translation expression, and proliferation will be examined while in TEPM ERK1/2 and SphK1, S1P levels, and transcription/translation of SphK1, IL10, IL6, TNF, IL1 (ELISA of secreted cytokines will be measured) will be explored. A third approach will utilize TEPM macrophages isolated from ERG1 KO male and female mice (Jackson laboratories) and treated with EMAP II.

In this macrophage study phosphorylation of ERK1/2 and SphK1, S1P levels (intracellular and secreted), and transcription/translation of SphK1, IL10, IL6, TNF, IL1 (ELISA of secreted cytokines will be measured) will be examined Caveats, alternative approaches and future directions: Our induced STATS phosphorylation and cytokine transcription were performed in recruited partially activated TEPM macrophages isolated from the peritoneum of C57BL6 mice. Similar studies were replicated in the RAW 264.7 murine macrophage cell line including the upstream ERK1/2 mediated transcription/translation of SphK1 and elevated S1P levels. However, it is possible that partially activated macrophages are not the most ideal macrophages to explore the S1P signaling pathway. Previously we have utilized undifferentiated bone marrow cells that when cultured in the presence of macrophage colony-stimulating factor demonstrate cell differentiation towards an M2 polarization (anti-inflammatory) phenotype. The anti-inflammatory phenotype of these cells could alter the impact that EMAP II has on the S1P signaling pathway. Future studies will explore whether EMAP II induces a different S1P signaling pathway in these bone-marrow-derived macrophages (BMDM) in cells isolated from SphK1 and ERG1 KO mice. If EMAP II is found to activate SphK2 phosphorylation, studies will explore the impact of chemical and SphK2 KO mice has on EMAP II mediated S1P expression.

### Example 3

### Identify the Mechanism of EMAP Transcriptional Regulation of SphK1 Through ERK1/2/EGR1.

Transient and sustained ERK signaling is known to induce a broad number of both early and late responding genes. As a result of this staggered signaling interaction, primary response genes initiated by ERK signaling mediate the transcription of secondary response genes that frequently have contrasting roles in crucial cell fate decisions such as proliferation and apoptosis. Based on literature and our preliminary data, we believe that EMAP II induces a staggered transcription of EGR1 as its phosphorylation of ERK1/2 results in its nuclear translocation followed by increased expression of a member of the zinc-finger family of transcription factors EGR1, and the subsequent EGR1 mediated SphK1 transcription. Similar to other studies linking EGR1 expression to SphK1 transcription, our data suggests that EMAP II induction of SphK1 transcription is regulated through an ERK1/2/EGR1 mechanism. Western blot (WB) and qPCR analysis are supportive of EMAP II's regulation of SphK1 transcription/translation while delivery of an EMAP II neutralizing antibody reduced pSphK1 and knockdown of EGR1 using siRNA inhibited EMAP II induction of EGR1. Our studies focus on determining if EMAP II signaling through ERK1/2 mediates EGR1 and SphK1 transcription as primary or secondary response genes, identification of the location of EGR1 translation due to its prominent nuclear expression, determine whether EGR1 is stabilized by post-translational modifications, and the relationship between EGR1 expression and SphK1 transcription/translation.

Impact of EMAP II on ERK1/2 Primary Response Gene EGR1/SphK1 Transcription and translation:

Previous studies link ERK1/2 mediated transcription of EGR1 as part of an ERK1/2 staggered signaling interaction. Our preliminary studies suggest that EMAP II transcriptional regulation of SphK1 is mediated through this step-wise mechanism. The transcription and expression kinetics of EMAP II using TEPM macrophages and hPASMC stimulated with EMAP II will be examined using an on/off scenario. To distinguish between EMAP II/ERK1/2 primary and secondary response genes, translation will be blocked with cycloheximide (CYHX) in parallel with EMAP II stimulation. Pulses of EMAP II initiated ERK1/2 signaling will be regulated using timed delivery of chemical ERK inhibitors (30 min before, 1 and 2 hours post EMAP II). Actinomycin D (ActD) will be added to determine if mRNA half-lives is mediated through transcriptional shut down. EGR1 and SphK1 mRNA (qPCR) and protein levels (nuclear and cytoplasmic) will be monitored over an 8-hour time course (0.5, 1,2,3,4,6, and 8 hrs). Blockade of EGR1 translation-using CYHX will determine if SphK1 transcription is a primary or delayed ERK1/2 transcription gene. To confirm EGR1 role in SphK1 transcription, some studies will utilize TEPM isolated from EGR1 KO mice and EGR1 knockdown in hPASMC using siRNA and analyzed as outlined above. Validation of EMAP II transcriptional regulation of EGR1 and SphK1 will be confirmed through analysis of EMSA and ChIP assays of target mRNAs.

### Identify Mechanisms of EMAP II Regulated EGR1 Translation.

Isolated nuclear and cytoplasmic protein fractions from EMAP II stimulated macrophages and hPASMC cells and immunofluorescence suggest that EGR1 is upregulated in the nuclear fraction. As protein synthesis is a dynamic process, it is unclear whether EGR1 is undergoes cytoplasmic translation and is shuttled back into the nucleus while residual cytoplasmic EGR1 is targeted for degradation, or if EGR1 is translated within the nucleus. Combining two methods that we have acquired expertise in (SUnSET/PURO and PLA), we will determine location and stability of EMAP II induced EGR1 expression. Global analysis of EGR1 translation will be explored using a puromycin (PURO) based labeling method that allows incorporation of puromycin into nascent peptides for fast and sensitive incorporation. Labeling of newly synthesized proteins with puromycin (SUnSET/ribopuromycylation) can be visualized using puromycin tagging and immunostaining techniques. In conjunction with PURO, the visualization of specific newly synthesized proteins will be observed using a proximity ligation assay (PLA) that detects spatial coincidence of a protein-specific antibody in correlation with the puromycin tag (PURO) or PURO-PLA. Using this mechanism, we can track EMAP II induction of EGR1 translation. EGR1 stability has been associated with its post-translation modifications of acetylation, phosphorylation, ubiquitination and sumoylation. To determine if post-translational modifications occur some studies will have the proteosomal inhibitor MG-132 added and evaluated using PURO-PLA and western blotting of nuclear and cytoplasmic isolates for EGR1, SphK1 and pSPHK1.

### Example 4

### Determine Impact of EMAP II on Intracellular SphK1 Trafficking and S1P Secretion.

SphK1 resides in the cytoplasm when upon stimulation through phosphorylation by ERK1/2 at Ser225, it translocates to the plasma membrane. Studies indicate that one of the predominate proteins facilitating relocation of SphK1 to the membrane is CIB1 (CaM-related calcium and integrin-binding protein 1). As a calcium-myristoyl switch protein, CIB1 provides a plasma membrane docking location via myristoylation where its retention facilitates sphingosine phosphorylation. Once SphK1 actively translocates to the plasma membrane, there is a noted increase in intracellular calcium levels as they are mobilized from the endoplasmic reticulum. The shift in intracellular calcium levels is consistent with previous reports that EMAP II treatment causes a redistribution of intracellular calcium stores into the cytosol supporting CIB1 involvement in SphK1 docking. Although a second CIB protein, CIB2 interacts with SphK1 in the same binding site as CIB1, it lacks the Ca2+-myristoyl switch function thereby blocking the docking of SphK1 to plasma membrane to interact with sphingosine. Our data supports an EMAP II mediated pSphK1 sphingosine S1P as EMAP II treated macrophage cells demonstrate a time dependent increase in S1P levels and a reduction in precursors sphingosine and ceramides. Furthermore, in hPASMC cells our preliminary data suggests that EMAP II treatment induces a bi-modal induction of S1P expression that parallel the pSphK1. These studies in collaboration with the Stahelin lab are designed to monitor the intracellular trafficking of SphK1 and S1P secretion.

Identify the Impact of EMAP II on the Intracellular Location of pSphK1:

Cytoplasmic trafficking of SphK1 in response to EMAP II will be determined in TEPM and hPASMC. Initial snapshots using timed studies will be done to confirm that EMAP II induces pSphK1 translocation to the cell membrane. Cells grown on chamber slides and exposed to EMAP II will be probed at 0 and 30 min and 1 hour for anti-pSphK1, anti-actin, and anti-cortactin antibodies to track pSphK1 location to the cell membrane. In parallel, cold-biotinylation of cells treated with EMAP II will assess membrane bound pSphK1. To confirm in a time dependent manner transport of SphK1 to the cell membrane, SphK1-TurboGFP tagged expression plasmid (Origene) will be overexpressed in hPASMC and trafficking of the protein monitored by spinning confocal microscopy following EMAP II treatment. Some studies will include ERK1/2 chemical inhibition to validate EMAP II ERK1/2 pSphK1 translocation. Images will be collected (using raster image correlation spectroscopy and number and brightness (N&B) analysis) in live cells on a Zeiss 880 microscope with a built in N&B analysis function (63×1.4 NA plan apochromat objective). Other studies will utilize a novel technique that couples chemical fixation and high-pressure freezing of cells with peroxidase tagging to allow precise localization of membrane proteins while conserving subcellular membrane architecture. For these studies, we will construct an SphK1-APEX2 plasmid (GenScript cDNA ORF clone) that will be transfected into hPASMC and cells will be exposed to EMAP II. Live cells will then be pelleted, fixed with glutaraldehyde, treated with DAB+hydrogen peroxidase and fixed by high pressure freezing. Following slow dehydration and staining by heavy metals, cells will be embedded in resin, sectioned, and SphK1 visualized by electron microscopy. Chemical inhibition of ERK1/2 will be performed in some studies.

### Example 5

### Materials and Methods

Materials. The primary antibodies STATS, pSTAT3 (Y705), ERK1/2, pERK1/2, SPHK1, EGR1, Lamin B1 and cyclin 1 were from Cell Signaling Technology (CST) (Danvers, Mass.), SPHK1 and alpha-tubulin was from Abcam and phospho-SPHK1 (Ser²²⁵) was purchased from ECM Biosciences. HRP-conjugated rabbit and mouse secondary antibodies were from CST, Alexa fluorescent labeled secondary antibodies and phalloidin were purchased from Thermo Fischer Scientific (Waltham, Mass.). Inhibitors W 146 (iS1PR1), JTE 013 (iS1PR2), TY 52156 (iS1PR3) and PF543 (iSPHK1) were from Cayman Chemical (Ann Arbor, Mich.), PD 184161 (iERK) was from Santa Cruz Biotechnology (Dallas, Tex.) and Ruxolitinib (iJAK1//2) and Trametinib (iERK1) were purchased from LC laboratories (Woburn, Mass.). EMAP II neutralizing antibody (Anti-EMAP II) was used to block EMAP II activity (Schwarz et al Mech Dev. 2000; 95(1-2):123-132.

### EMAP II Preparation

6x-His tagged EMAP II was prepared as previously described (Mech Dev. 2000. doi:10.1016/S0925-4773(00)00361-0). The endotoxin levels in all preparations used for this study were below detectable limits, containing<0.1 ng/EU (GenScript).

### Cell Culture and Treatments

RAW 264.7 (ATCC) mouse macrophages and HEK-293T (ATCC) cells were cultured in DMEM media with 10% (v/v) fetal bovine serum (FBS) and 2 mM glutamine HEPES buffer and beta-mercaptoethanol were added to the growth medium used for RAW 264.7 macrophages. Primary human pulmonary artery smooth muscle cells (hPASMC) purchased from Lonza (Walkersville, Md.) were cultured in complete growth medium containing smooth muscle growth media-2 (SmGM-2) with 10% FBS and growth factors provided as a kit by the supplier (Lonza, (Walkersville, Md.). THP-1 human monocyte cells were cultured in RPMI 1640 with 10% FBS and glutamine Cells were cultured in a humidified atmosphere with 5% CO₂ at 37° C. For all studies passages 5-14 were used for RAW 264.7 and passages 5-10 were used for hPASMCs. For treatment studies, subconfluent cells plated in multi-well plates were serum starved and if required, pretreated with inhibitors before stimulation with 2 μg/mL recombinant EMAP II.

### Mouse Lung Tissues

Animal experimental procedure was performed in accordance with the guidelines issued by the University of Notre Dame/Indiana University Institutional Animal and Use Committee. Lung tissues were harvested from postnatal day 15 C57BL/6 mice (Jackson laboratories) that were injected with either EMAP II or PBS at Day 3. Total protein was extracted and quantified. 1 mg of total protein was subjected to S1P quantification by HPLC/MS/MS.

### ELISA for S1P Quantification

Equal number of hPASMC cells (1,000,000) were plated and serum starved for overnight. Cells were stimulated for in a series of time points and collected the cells. Cells were pelleted out and washed with PBS. After the lysis protein concentration was measured. S1P levels were measured using S1P ELISA kit (MyBioSource, MBS069092) following manufacturer's instructions. S1P levels were normalized against protein levels.

### Analysis of S1P, Sphingosine and Ceramides by LC-MS/MS

Collected cells were rinsed with PBS, pelleted. Cell pellets and 1 mg of protein tissue extracts were frozen at −80° C. Sphingolipids extraction and analysis was carried out at the Medical University of South Carolina (MUSC) core facility using eight-point calibration curves generated for each target analyte by High Performance Liquid Chromatography-Tandem Mass Spectrometry (HPLC/MS/MS) system operating in positive multiple reaction-monitoring (MRM) mode engaging a gradient elution. Sphingolipids were normalized to inorganic phosphate levels in PASMC which was determined from the Bligh & Dyer lipid extraction method. Biological samples were from different passages collected on different days.

### Immunoblotting

After appropriate treatments, protein lysates were prepared by using RIPA buffer supplemented with phosphatase and protease inhibitors (Thermo Fisher Scientific). Alpha-tubulin was used as a loading control. For nuclear and cytoplasmic fractionation, NE-PER kit from Thermo Fischer Scientific was used. Western blots were performed according to standard methods and quantified using densitometry using Image Studio Lite software from LI-COR Biosciences.

### Transfection and Luciferase Assay

HEK 293-T cells cultured in DMEM media containing 10% serum were reverse co-transfected with pGL4.47fluc2P/SIE/Hygrol and pGL4.74 [hRluc/TK] using Attractene Transfection Reagent (301005, Qiagen) as per manufacturer's protocol. Pretreated with inhibitors for 1 hour as required. After the treatment cells were incubated for 24 hours and luciferase activity was measured using Dual-Glo Luciferase Assay (E2920, Promega) per manufacturer's guidelines.

### Differentiation of THP-1 Cells

Few passages after thawing of THP-1 cell line, cells were seeded in Nunc Lab-Tek™ II 4-well imaging plates and differentiated into macrophages with 100 nM Phorbol myristate acetate (InvivoGen, CA) for 72 hours.

### Immunofluorescence Staining

Primary hPASMCs cells or human THP-1 macrophages were seeded in Nunc Lab-Tek™ II 4-well imaging plates (Thermo Fisher Scientific) and cultured in growth medium. After the serum starvation cells were stimulated with EMAP II. Treated cells were rinsed with PBS and fixed with 4% paraformaldehyde at room temperature for 15 minutes and permeabilized with 0.1% Triton X-100 at room temperature for 10 min. After washing with PBS three times, the cells were incubated with EGR1, 1amin, or pSPHK1 antibody at 4° C. overnight. The cells were then rinsed with PBS three times and subsequently incubated with respective secondary antibody conjugated with Alexa Fluor 647 and 488 or Phalloidin 488 at room temperature for 1 h. The cells were rinsed with PBS three times and coverslips were mounted with SlowFade Gold Antifade Mountant with DAPI (Thermo Fischer Scientific) and the cells were examined under Olympus microscope with 40× water objective lens for EGR1 expression and with 60× for pSPHK1 water immersion.

Transfection with Small Interfering RNA

Knockdown of endogenous EGR1 was carried out by transfecting with 25 nM final concentration of ON-TARGETplus siRNAs specific for EGR1 or non-targeting control (GE Dharmacon, Lafayette, Colo.) using Viromer Blue (Lipocalyx) per manufacturer's instructions. Cells were stimulated with EMAP II 48 h post-transfection.

### Cell Proliferation Assay

Cell proliferation of hPASMCs was assessed using WST-1 cell proliferation reagent (Roche Diagnostic Corporation, IN) in a 96-well plate. Human PASMCs were seeded at 4200 cells/well density in 100 μl complete medium and serum starved for overnight. Cells were stimulated for 48 hours with 2 μg/mL EMAP II following the pretreatment with inhibitors. WST-1 reagent (10 μL) was added into each well and incubated for 90 minutes in a humidified atmosphere with 5% CO₂ at 37° C. Absorbance was measured at 450 nm using microplate reader. Blank control well was with medium and WST-1 reagent without cultured cells. All samples were in duplicates. Cell proliferation was determined by subtracting the background reading from average of duplicate of each sample.

### RNA Extraction and Quantification

Total RNA was extracted with TriZol using standard RNA extraction protocol and reverse transcribed with SuperScript III Reverse Transcriptase (Thermo Fisher Scientific). SPHK1, Tnf, Il6, EGR1, Gapdh, Hprt1 transcripts were quantified using PrimePCR SYBR Green assays from BioRad.

### Statistical Analysis

The data are presented as means±1 standard deviation or standard error of mean from at least three independent experiments. Statistical significance was determined with unpaired Student's t-test or one-way ANOVA using GraphPad Prism software.

### Results

### EMAP II Modulates S1P Levels In Vitro and In Vivo.

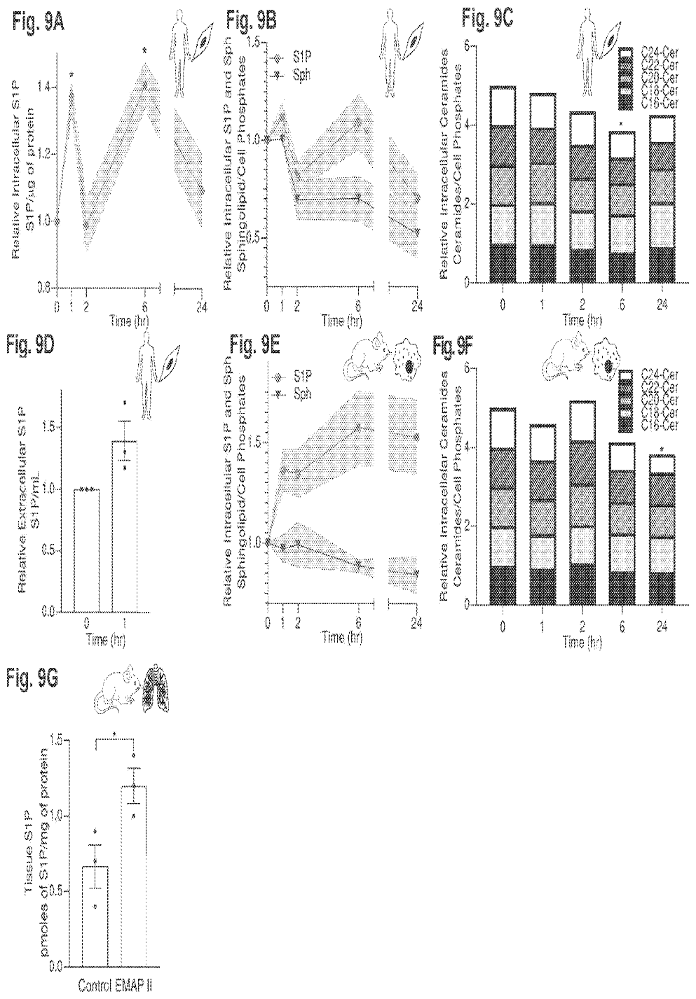

EMAP II mediates a distinct inflammatory gene profile in recruited macrophages through a JAK/STAT signaling pathway similar to S1P signaling axis. Moreover, our previous studies reported EMAP II promoted secondary PAH in BPD like phenotypic mouse model. Therefore, we investigated the potential of EMAP II to modulate S1P levels in two major cell populations in PAH, the pulmonary arterial wall component PASMCs and a circulating major inflammatory component, macrophages. Following stimulation by EMAP II in a time course, intracellular SIP levels of smooth muscle cells (SMCs) measured by ELISA peaked at 1 hour, subsided at 2 hours peaked again at the 6 hour time point before subsiding at 24 hour exhibiting a significant bimodal pattern in S1P elevation (FIG. 9A). Examination of SIP precursors and metabolites by HPLC-MS/MS revealed cellular sphingosine and ceramide levels demonstrated decreasing levels over time both in SMCs and macrophages after EMAP II treatment (normalized against inorganic phosphates) (FIGS. 9B, 9C, 9E and 9F). In line with ELISA studies, the cellular S1P measurements in both SMCs and macrophages by HPLC/MS/MS showed a similar bimodal trend (FIGS. 9B and 9E). Extracellular S1P in SMCs were elevated after 1 hour of EMAP II treatment compared to the control (FIG. 9D) suggesting the possibility of S1P acting as an extracellular ligand for S1P receptors.

Moreover, delivery of recombinant EMAP II subQ to neonatal mice on days of life 3-15, previously shown to induce distal pulmonary artery thickening and right ventricular hypertrophy consistent with pulmonary hypertension, significantly increased whole lung S1P levels as assessed by HPLC/MS/MS compared to the control (FIG. 9G).

EMAP II Induced STAT3 Activation in Macrophages is Linked with S1P Signaling and ERK activation.

Our previous studies demonstrated that EMAP II induces a JAK dependent biphasic tyrosine phosphorylation of STAT3 (Y705) and increases STAT3 mediated transcriptional target genes in macrophages. Therefore, in this study, we explored the sequential steps and contribution of S1P signaling axis linked to EMAP II signaling. EMAP II induced STAT3 activation in macrophages while pretreatment with S1PR specific inhibitors [iS1PR1 (S1PR1 inhibitor, 1 μM), iS1PR2 (S1PR2 inhibitor, 1 μM), iS1PR3 (S1PR3 inhibitor, 1 μM)], SPHK1 inhibitor [iSPHK1, 100 nM)] as well as a JAK1/2 inhibitor [iJAK1/2, 1 μM] significantly inhibited EMAP II induction of pSTAT3 (FIGS. 10A and 10B). These findings were further supported as luminescence of STAT3 transcription reporter plasmid (FIG. 10C) demonstrated that EMAP II induced STAT3 activation is linked to the S1P signaling axis.

Pharmacological inhibition of SPHK1 significantly inhibited EMAP II induced STAT3 activation and luminescence of STAT3 transcription reporter plasmid (FIGS. 10A, 10B and 10C) suggesting that EMAP II signaling was upstream of S1PR. In SMCs, no STAT3 activation was observed (data not shown). As EMAP II is a promiscuous mediator of tyrosine kinase signaling, we tested the impact of EMAP II on the activation of a common tyrosine kinase-signaling factor, ERK1/2. Similar to EMAP II induction of ERK1/2 phosphorylation in endothelial cells22, EMAP II induced ERK1/2 phosphorylation in macrophages (FIGS. 11A-11F) while pretreatment with a chemical inhibitor to block phosphorylation of ERK1/2 (iERK: MEK inhibitor, 10 μM) significantly inhibited EMAP II induced pSTAT3 (FIGS. 10D and 10E) suggesting that pSTAT3 activation may occur through EMAP II initiated ERK1/2 mediated signaling cascade.

EMAP II Induces Time Dependent Bimodal ERK Activation and Overexpression of immediate early transcription factor EGR1.

To determine the impact and timing of EMAP II induced ERK1/2 phosphorylation, a 24-hour time course examined the impact of EMAP II on pERK1/2 in two known S1P signaling cellular targets, macrophages and SMCs. We identified a time dependent statistically significant bimodal activation of pERK1/2 (normalized to ERK1/2) in macrophages (FIGS. 11A and 11B) as early as 5 minutes and reached the peak at 1 hour before subsiding only to again be noted to increase at 24 hours. EMAP II induced pERK1/2 in SMCs in a similar time dependent manner (FIGS. 11D and 11E) where pERK1/2 reached significance as early as 3 minutes, subsided and steadily started increasing back again at 24 hours. Since EMAP II signaling is intertwined around both ERK activation (FIGS. 10D, 10E, 11A, 11B, 11D and 11E) and SPHK1 (FIGS. 10A, 10B and 10C), we explored the effect of EMAP II on an immediate early transcription factor, EGR1 which has SPHK1 as a target gene 31,32 and is known to be transcriptionally mediated by ERK1/2 33,34. Protein levels of EGR1 in whole cell lysates from time course studies in both macrophages and SMCs demonstrated a significant upregulation of EGR1 expression at 1 to 2 hours following EMAP II treatment in macrophages and SMCs (FIGS. 11A, 11C, 11D and 11F).

### EMAP II Induced Upregulation of Nuclear EGR1 Expression is Mediated Through ERK Activation.

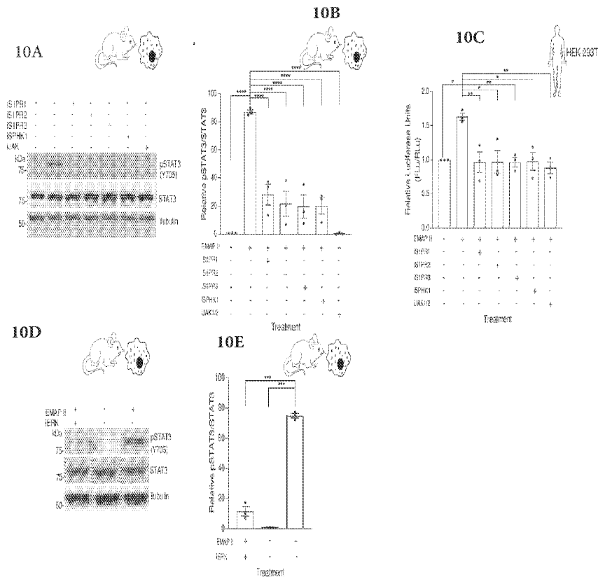

Previous studies have identified nuclear pERK1/2 as an important transcriptional regulator of EGR1 35. Utilizing nuclear and cytoplasmic fractions, the impact of EMAP II stimulation on pERK1/2 and EGR1 nuclear expression in macrophage and SMC populations was studied. Converse to pERK1/2 mediated EGR1 transcription, MEK inhibition by iERK (MEK inhibitor, 10 μM) and iERK1 (MEK inhibitor, 10 μM) blocked EMAP II induced EGR1 protein expression in both macrophages (FIGS. 12A and 12B) and SMCs (FIGS. 12 D and 12E). To determine whether EGR1 transcription is mediated through ERK activation, EGR1 transcripts were quantified in macrophages and SMCs with or without iERK1 (MEK inhibitor, 10 μM) pretreatment following EMAP II stimulation and normalized against Hprt1 in macrophages (FIG. 12C) and Gapdh in SMCs (FIG. 12F). Results demonstrated EMAP II induced EGR1 transcription can be inhibited by blocking ERK1/2 phosphorylation. Furthermore, EMAP II significantly (9-fold) upregulated nuclear expression of EGR1 in macrophages at 2 hours (FIGS. 13A and 13B) and in SMCs, EMAP II upregulated EGR1 expression (2.5-fold) at 1 hour before subsiding (FIGS. 13C and 13D) Immunofluorescent analysis of SMCs stimulated with EMAP II for 1 hour demonstrated significantly elevated nuclear EGR1 expression as indicated by co-localization of EGR1 (red), DNA (blue) and 1amin (green) as compared to control (FIGS. 13E and 13F). These results suggest that EMAP II upregulated EGR1 play a major role as a transcription factor in EMAP II mediated signaling cascade.

### EMAP II Upregulates SPHK1 Transcription and Translation Through EGR1.

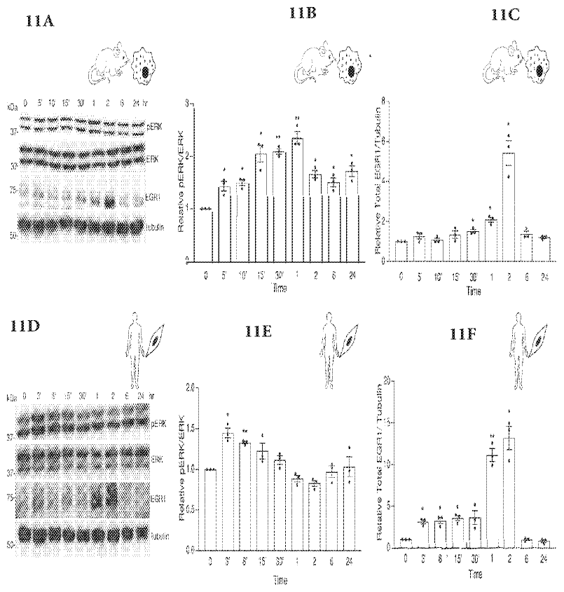

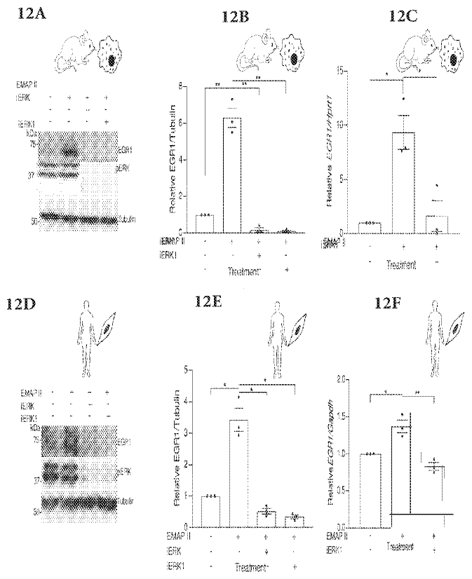

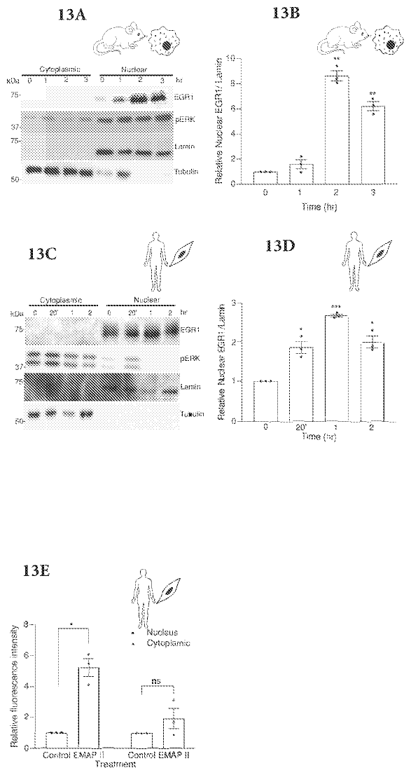

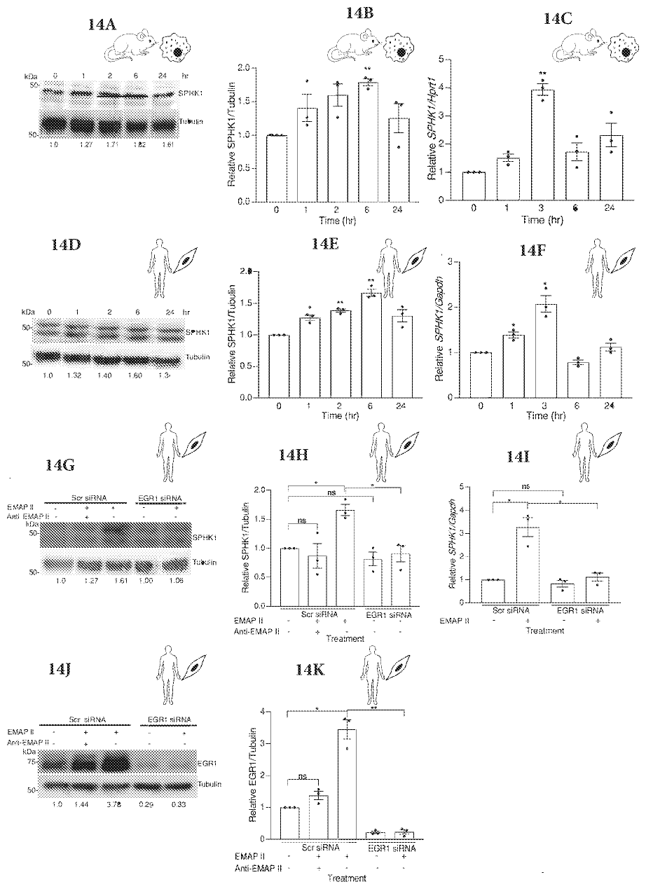

As EGR1 has been previously identified as a SPHK1 transcriptional mediator, the effect of EMAP II signaling on SPHK1 expression was measured. EMAP II significantly increased expression of SPHK1 at 6 hours in both macrophages (FIGS. 14A and 14B) and SMCs (FIGS. 14D and 14E). To determine the effect of EMAP II on SPHK1 transcription, qRT-PCR of SPHK1 transcription was performed. EMAP II induced a time-dependent increase in SPHK1 transcription that peaked at 3 hours after EMAP II stimulation in both macrophages and SMC types (FIGS. 14C and 14F).

Furthermore, knockdown of EGR1 using specific siRNA and blocking EMAP II activity by pretreating with an EMAP II neutralizing antibody significantly attenuated EMAP II induced SPHK1 protein upregulation at 6 hours (FIGS. 14G and 14H) and upregulation of transcript level at 3 hours (FIG. 14I). Successful knockdown of EGR1 was confirmed by immunoblotting (FIGS. 14J and 14K).

### EMAP II Induces Bimodal Phosphorylation and Plasma Membrane Localization of SPHK1.

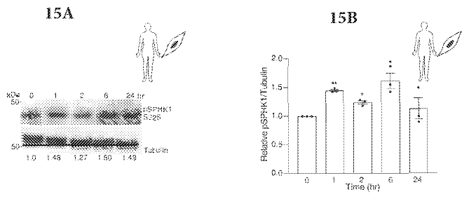

Having established that SPHK1 is upregulated by EMAP II through EGR1, we sought to determine whether EMAP II affects the activation of SPHK1. Since phosphorylation of SPHK1 at Ser225 is required for SPHK1 activity 36, we initially studied the pSPHK1 protein expression after EMAP II stimulation at specific time points. In line with EMAP II induced bimodal S1P peaks, immunoblotting demonstrated that EMAP II induced bimodal SPHK1 phosphorylation at 1 and 6 hours in SMCs (FIGS. 15A and 15B). SPHK1 resides in the cytoplasm where upon phosphorylation by ERK1/2, it translocates to the plasma membrane 36. Consistent with the immunoblotting data, immunofluorescence showed increased pSPHK1 signal (red) co-localized with actin (green) staining boundaries in SMCs stimulated with EMAP II for 15 minutes yet persisted for 60 minutes (FIG. 15C). Similar to SMCs, THP-1 macrophages also exhibited a similar a response to SMCs; elevated levels of pSPHK1 was observed near the cell membrane at 1 hour of EMAP II stimulation (FIG. 15D) as indicated by actin staining. In addition, we observed that in SMCs after 6 hours of EMAP II treatment, pSPHK1 is concentrated around the membrane (FIG. 15E) demonstrating bimodal phosphorylation of SPHK1 at 1 and 6 hours as suggested by the immunoblotting for pSPHK1.

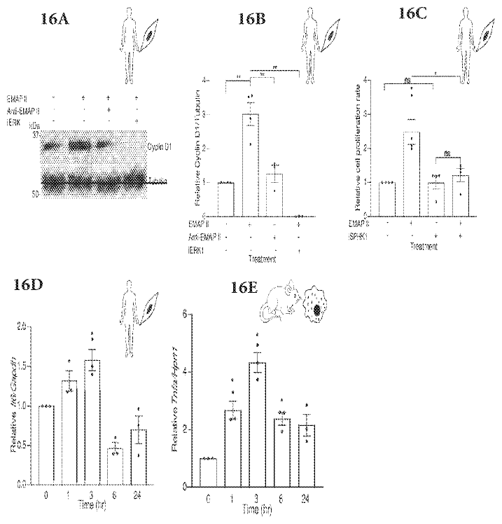

The downstream effects of EMAP II/SIP signaling are cell type specific. Although EMAP II signaling activates STATS in macrophages as shown in FIGS. 10A-10E, EMAP II does not impact pSTAT3 levels in SMCs (data not shown). Moreover, EMAP II induced tumor necrosis factor (TNF) cytokine production in macrophages as demonstrated by increased Tnf transcription level (FIG. 16E) while this observation was absent in SMCs (data not shown). We discovered that EMAP II modulates cyclin D1 in SMCs in an ERK1/2 dependent manner instead of TNF□ after 48 hours of EMAP II stimulation (FIGS. 16A and 16B) which was confirmed by WST1 proliferation assay (FIG. 16C) suggesting that EMAP II promotes cell proliferation in SMCs. Importantly, inhibition of SPHK1 ablated EMAP II promoted proliferation in SMCs (FIG. 16C) without affecting the basal cell proliferation rate. Likewise, increased transcription of a major proliferative marker in SMCs 37 interleukin 6 (I16) was found in SMCs by qRT-PCR (FIG. 16D).

### Discussion

The regulatory role of the bioactive lipid S1P in vasoconstriction, proliferation, fibrosis and vascular inflammation has attributed a growing recognition of S1P as a critical regulator of several cardiovascular and pulmonary pathophysiological processes including PAH. Understanding the molecular mechanism and key regulators of enigmatic S1P could guide the discovery of potential treatments for PAH. The molecular regulation of S1P generation catalyzing kinase SPHK1 is not well described. Here, we report a novel role of EMAP II in mediating cellular responses through triggering a bimodal phosphorylation of sphingosine, generating S1P in a two-pronged manner by modulating phosphorylation, transcriptional regulation and translocation of SPHK1 through a common and coherent upstream signaling in inflammatory cells and SMCs. EMAP II initiates cell specific downstream pathophysiological functions that may contribute to the pathogenesis PAH.

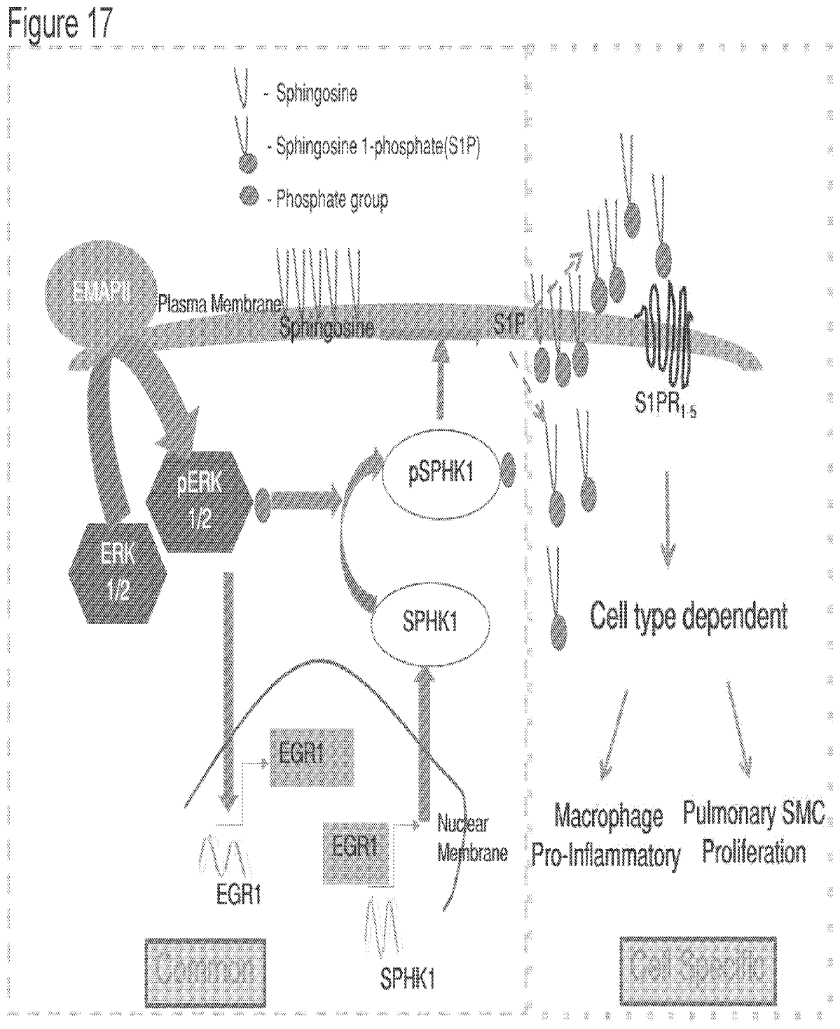

We identified that 1) EMAP II induces bimodal ERK1/2 activation and SPHK1 activation, 2) EMAP II induced ERK1/2 results in overexpression of transcription regulator of differentiation, inflammation, and mitogenesis protein coding gene EGR1, 3) EMAP II induced elevated nuclear expression of EGR1 regulates SPHK1 transcription, and 4) EMAP II promotes membrane localization of pSPHK1 and increases S1P expression (FIG. 17). Thus, identifying the upstream crosstalk between S1P and EMAP II in downstream molecular and signaling consequences that stimulate macrophage pro-inflammatory mediators and SMC proliferation associated with development PAH sheds new insight into the regulation of S1P homeostasis.

S1P has been implicated in several cardiopulmonary diseases. S1P levels reported to be elevated in lung tissues of patients with PAH where increased transcription of SPHK1 showed to play the critical role in disrupting the S1P homeostasis. SPHK1 mediated increased proliferation of perivascular SMCs reported to cause PAH. Moreover, a clinical study identified S1P as a stronger biomarker of severe atherosclerosis. Recent studies reported the knockdown or pharmacological inhibition of SPHK1 attenuates bronchopulmonary dysplasia (BPD) in mouse models warranting that SPHK1/S1P would be a potential signaling pathway to be targeted in BPD. Similarly, moonlighting roles for EMAP II in disease progression mediated by inflammation include mediating inflammation by immune cell trafficking, angiogenesis and apoptosis. Previous studies link EMAP II to endothelial cell apoptosis, a detrimental factor in atherosclerosis and increased expression of E- and P-selectins and Tnf-1 which results in monocyte and neutrophils attraction 46,47. Moreover, EMAP II mediated macrophage recruitment and pro-inflammatory cytokine production are seen in BPD mouse models. However, this study is the first to report the potential mechanistic contribution of EMAP II to the development of PAH. Both immunochemistry and mass spectrometry studies showed EMAP II stimulated bimodal peaking of S1P at 1 and 6 hours suggesting that the protein, EMAP II is a potential upstream mediator of S1P generation. Moreover, the depreciation of ceramides indirectly supports the observation of S1P elevation as they are interconvertible lipids and ceramide-sphingosine-S1P rheostat is known to control the cell fate 61. In line with bimodal peaking of SIP, immunoblotting and immunofluorescence studies of S1P generation catalyzing enzyme SPHK1 showed bimodal activation. The phosphorylation of SPHK1 at Ser225 by ERK1/2 activators such as phorbol esters and PDGF increases enzyme activity by 14-fold and mediates the translocation of the enzyme from the cytosol to the plasma membrane bringing it into proximity to its substrates.

We observed a similar bimodal pattern in both ERK1/2 and SPHK1 activation, suggesting an inter-dependent relationship between ERK1/2 and SPHK1. ERK1/2 is one of the members of mitogen-activated protein kinase super family and is known to directly phosphorylate SPHK1 at Ser225 residue. Additionally, SPHK1 carries a docking site for ERK1/2. However, there is evidence showing that ERK1/2 activated SPHK1 is capable of activating ERK1/2 back in a positive feedback mechanism suggesting that the early observed (1 hr) pSPHK1 may be the activator of secondary ERK1/2 activation in EMAP II/SPHK1/S1P signaling pathway. For the first time, we report that EMAP II phosphorylates SPHK1 at Ser225 by acting as an activator of ERK1/2 which may eventually initiate the feedback loop between ERK1/2 and SPHK1.

EMAP II induces overexpression of nuclear EGR1, another known target of ERK1/2 and transcription activator of SPHK1. EGR1 is a 80-82-kD inducible zinc finger transcription factor that has also been known to regulate cell growth, proliferation and inflammation in lung disorders. Identified as one of the early biomarkers of lung injury in preterm lambs, EGR1 mRNA levels were increased 120-fold within 30 minutes of injury. Moreover, pharmacological inhibition of EGR1 has been reported to attenuate pulmonary vascular resistance and right ventricular hypertrophy in animal models. Our experiments indicate that inhibition of EGR1 suppresses EMAP II mediated SPHK1 transcription representing a potential mechanism of regulating downstream S1P generation. Since our studies show that EMAP II signaling provokes a bimodal early upstream SPHK1 phosphorylation in addition to the later EGR1 mediated SPHK1 transcription and activation, it is questionable whether inhibition of EGR1 alone would be a promising therapeutic target for impeding EMAP II/SPHK1/S1P signaling in PAH. However, ablating ERK1/2 phosphorylation has been reported to inhibit EGR1 induced pulmonary vascular proliferation in newborn pulmonary hypertension calves models supporting the fact that the phosphorylation of ERK1/2 is a key regulator of both SPHK1 phosphorylation and transcriptional regulation. Thus providing a critical target point in regulation of downstream sphingosine phosphorylation. In addition, we observed about 4 times induction of SPHK1 transcription in EMAP II treated macrophages in compared to the control. Nevertheless, the protein expression of SPHK1 in macrophages was only about 1.7 times. One possibility for the difference between the fold change of transcripts and protein expression levels might be due to multiple factors that affecting the protein translation including the level of mRNA and its half-life, translational efficiency, and the stability of the interested protein.

To demonstrate the potential of translating this novel signaling mechanism into in vivo system, EMAP II treated postnatal day 15 mice were used as in Lee et al. 2016 reported right ventricular hypertension and increased elastin deposition in neonatal mice of postnatal day 42 treated with EMAP II in 3-15 days of life that promoted BPD-like phenotype suggesting that EMAP II induces secondary PAH signs associated with BPD. Moreover, Lee et al. 2016 reported that those mice exhibit high inflammatory responses on postnatal day 15. Compared to control, EMAP II treated postnatal day 15 mice lungs showed a significant increase in the tissue S1P levels suggesting that EMAP II induced S1P could contribute to the development of PAH in BPD.

Although upstream signaling of EMAP II/ERK/EGR1/SPHK1/S1P is common in both cell types of macrophages and SMCs, our studies demonstrated cell type-specific downstream responses suggesting that downstream effects may depend on the cell type and the availability of different S1PRs. In macrophages, EMAP II signaling induces STATS activation via S1PRs which mediates inflammatory state as shown by prior studies 17,18 while in SMCs, EMAP II signaling results in proliferative signaling by overexpressing cell cycle regulating proteins like cyclin D1 which is required for G1/S phase transition. We report that EMAP II promotes proliferation in SMCs challenging the conventional known apoptotic function of EMAP II in endothelial cells and is consistent with previous reports of increased peri-vascular SMC EMAP II expression in infants with BPD. A recent study has shown that macrophage migration inhibitory factor upregulates cyclin D1 via ERK1/2 leading to SMC proliferation causing pulmonary hypertension in animal models.

As such our future studies will explore if S1P/S1PR is a potential therapeutic target to regulate these downstream effects while minimizing consequences associated with S1P signaling. Elevated early response signaling molecules recruit inflammatory cells such as neutrophils and macrophages. Macrophages are the prominent inflammatory cells in chronic inflammation due to its functions in promotion and resolution of inflammation, pathogen clearance, and tissue restoration following injury 69. Chronic inflammation mediated by macrophages can contribute to the arterial remodeling observed PAH. The dynamic interplay between macrophages and SMCs in vascular diseases is well recognized. Therefore, the source of EMAP II that acts on SMCs to promote proliferation can be expressed and secreted in recruited macrophages suggesting a novel aspect of a complex signaling mechanism to be explored. We report EMAP II as a mediator of S1P burden. However, the proteolytically cleaved protein EMAP II and the bioactive lipid S1P are signals that get triggered as early response chemokines prior to influx of inflammatory cells upon the cellular damage. Therefore, in physiological conditions although it is possible that SIP is activated prior to EMAP II, our findings suggest that there is an intimate crosstalk between two biological molecules may initiate a feedback loop mechanism elevating cellular S1P concentration.

In summary, our findings indicate that EMAP II can be a potential key mediator of the bioactive lipid S1P. EMAP II modulated S1P signaling promotes activation of key mechanistic signaling pathways that can contribute to the pro-inflammation and SMC hyperproliferation in PAH through a two-phase process. An upstream common protein initiated biphasic activation of the bioactive lipid S1P and a lipid mediated downstream cell specific signaling pathway that contributes to PAH progression.

1. A method of treating an inflammatory disorder in a subject, the method comprising the step of:
administering to the subject in need thereof an endothelial monocyte activating polypeptide II (EMAP II) inhibitor.

2. The method according to claim 1, wherein the inflammatory disorder is a chronic respiratory disorder

3. The method according to claim 1, wherein the inflammatory disorder is bronchopulmonary dysplasia.

4. The method according to claim 1, wherein the EMAP II inhibitor comprises an EMAP II signaling pathway inhibitor, a blocking antibody, an Erk inhibitor and/or an EGR1 inhibitor.

5. The method according to claim 1, wherein the EMAP II inhibitor comprises an antibodies that specifically binds to EMAP II.

6. The method according to claim 1, wherein the EMAP II inhibitor is a compound having the structure:

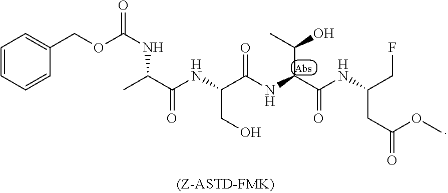 [SMILES: COC(=O)C[C@@H](CF)NC(=O)[C@@H](NC(=O)[C@H](CO)NC(=O)[C@H](C)NC(=O)OCc1ccccc1)[C@@H](C)O]

7. A method of treating a smooth muscle proliferation disorder in a subject, the method comprising the step of:
administering to the subject in need thereof an endothelial monocyte activating polypeptide II (EMAP II) inhibitor.

8. The method according to claim 7, wherein the smooth muscle cell proliferation disorder is be a vascular disorder such as atherosclerosis, restenosis, and pulmonary hypertension.

9. The method according to claim 7, wherein the EMAP II inhibitor comprises an EMAP II signaling pathway inhibitor, a blocking antibody, an Erk inhibitor and/or an EGR1 inhibitor.

10. The method according to claim 7, wherein the EMAP II inhibitor comprises an antibodies that specifically binds to EMAP II.

11. The method according to claim 7, where the EMAP II inhibitor comprises a compound having the structure: 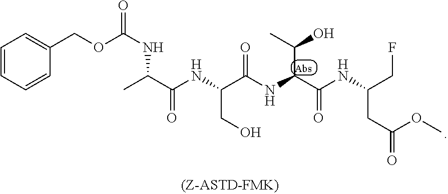 [SMILES: COC(=O)C[C@@H](CF)NC(=O)[C@@H](NC(=O)[C@H](CO)NC(=O)[C@H](C)NC(=O)OCc1ccccc1)[C@@H](C)O]


SEQUENCE LISTING (10 sequences)

SEQ ID NO: 1 - protein, 312 aa, Homo sapiens
MANNDAVLKRLEQKGAEADQIIEYLKQQVSLLKEKAILQATLREEKKLRVENAKLKKEIEELKQELIQAEIQNGVKQIPFPSGTPLHANSMVSENVIQSTAVTTVSSGTKEQIKGGTGDEKKAKEKIEKKGEKKEKKQQSIAGSADSKPIDVSRLDLRIGCIITARKHPDADSLYVEEVDVGEIAPRTVVSGLVNHVPLEQMQNRMVILLCNLKPAKMRGVLSQAMVMCASSPEKIEILAPPNGSVPGDRITFDAFPGEPDKELNPKKKIWEQIQPDLHTNDECVATYKGVPFEVKGKGVCRAQTMSNSGIK

SEQ ID NO: 2 - protein, 172 aa, Homo sapiens
IAGSADSKPIDVSRLDLRIGCIITARKHPDADSLYVEEVDVGEIAPRTVVSGLVNHVPLEQMQNRMVILLCNLKPAKMRGVLSQAMVMCASSPEKIEILAPPNGSVPGDRITFDAFPGEPDKELNPKKKIWEQIQPDLHTNDECVATYKGVPFEVKGKGVCRAQTMSNSGIK

SEQ ID NO: 3 - RNA, 19 nt, Homo sapiens
gaugaacgcaagaggcaua

SEQ ID NO: 4 - RNA, 19 nt, Homo sapiens
cgacagcagucccauuuac

SEQ ID NO: 5 - RNA, 19 nt, Homo sapiens
ggacaugacagcaaccuuu

SEQ ID NO: 6 - RNA, 19 nt, Homo sapiens
gaccugaaggcccucaaua

SEQ ID NO: 7 - RNA, 19 nt, Homo sapiens
ugguuuacaugucgacuaa

SEQ ID NO: 8 - RNA, 19 nt, Homo sapiens
ugguuuacauguuguguga

SEQ ID NO: 9 - RNA, 19 nt, Homo sapiens
ugguuuacauguuuucuga

SEQ ID NO: 10 - RNA, 19 nt, Homo sapiens
ugguuuacauguuuuccua